(19) United States
(12) Patent Application Publication
(10) Pub. No.: US 2025/0027049 A1
(43) Pub. Date: Jan. 23, 2025

(54) ISLET-LIKE ORGANOID EXPRESSING MONSTIM1 IN WHICH INSULIN SECRETION IS REGULATED BY OPTICAL STIMULATION

(71) Applicant: KOREA ADVANCED INSTITUTE OF SCIENCE AND TECHNOLOGY, Daejeon (KR)

(72) Inventors: Yong Mahn Han, Daejeon (KR); Wondo Heo, Daejeon (KR); Ji Eun Choi, Daejeon (KR); Jinsu Lee, Daejeon (KR); Eun Ji Shin, Daejeon (KR)

(73) Assignee: KOREA ADVANCED INSTITUTE OF SCIENCE AND TECHNOLOGY, Daejeon (KR)

(21) Appl. No.: 18/715,678

(22) PCT Filed: Nov. 29, 2022
(86) PCT No.: PCT/KR2022/019109

§ 371 Date: May 31, 2024

(30) Foreign Application Priority Data
Dec. 2, 2021 (KR) 10-2021-0170587
Apr. 20, 2022 (KR) 10-2022-0048550

(51) Int. Cl. C12N 5/071; A61K 35/39; A61P 3/10

(52) U.S. Cl. CPC C12N 5/0677; A61K 35/39; A61P 3/10; C12N 2506/02; C12N 2506/45; C12N 2510/00; C12N 2529/10

(57) ABSTRACT

The present invention relates to an islet-like organoid in which insulin secretion can be optically regulated by differentiating, into pancreatic islets, human pluripotent stem cells (monSTIM1-hPSC) obtained by knocking-in monSTIM1, which is an inducer of Ca²⁺ concentration increase (Ca²⁺ transient), into the AAVS1 locus of the stem cells. Since it has been confirmed in vitro and in the body of a diabetic mouse model that the islet-like organoid of the present invention can secrete insulin through an intracellular calcium influx increased by light irradiation, the islet-like organoid can be effectively used in the treatment of diabetic patients.

### BACKGROUND OF THE INVENTION

### 1. Field of the Invention

The present invention relates to an islet-like organoid in which insulin secretion is regulated by light irradiation.

### 2. Description of the Related Art

Calcium (Ca²⁺) is critical for cellular function and is widely involved in cell motility, division, gene expression, neurotransmitter secretion, and homeostasis. In order for cells to perform their functions well, the intracellular calcium ([Ca²⁺]ᵢ) concentration must be properly regulated. The calcium-initiated signaling pathway is determined by calcium oscillations, frequency, amplitude, and duration, as well as spatial factors that indicate where in the cell the rapid transient increase in Ca²⁺ concentration occurs.

Optogenetic approaches can be used to regulate the release of calcium. Optogenetics is a biological technology that can control cells in living tissue with light. A representative example is genetically manipulating nerve cells to express ion channels that respond to light. Using optogenetics, it is possible to control and observe the activity of biological tissues and individual nerve cells. The main material needed for optogenetics is a protein that responds to light. Optogenetic actuators such as channelrhodopsin, halorhodopsin, and achirhodopsin can be used to regulate neural activity, and optogenetic sensors such as GCaMP to detect changes in calcium concentration, synaptopHluorin to detect secretion of neuroendoplasmic reticulum, GluSnFRs to detect neurotransmitters, and Arclightning (ASAP1) to detect cell membrane potential can be used to record neural activity optically and visually. Therefore, using optogenetics, there is the possibility to control the rapid intracellular Ca²⁺ concentration increase (Ca²⁺ transient) by regulating intensity, time, and space.

Examples of the proteins that respond to light include OptoSTIM1 (Nat Biotechnol. 2015 October; 33(10):1092-6.) and monSTIM1 (NATURE COMMUNICATIONS, (2020) 11:210), which has a 55-fold increased sensitivity to light compared to OptoSTIM1. The mechanism of action of OptoSTIM1 and monSTIM1 is based on store-operated Ca²⁺ entry (SOCE), which is mediated through the binding of stromal interaction molecule 1 (STIM1) to calcium release-activated calcium channel protein 1 (CRAC), the pore-forming unit of the Ca²⁺-release-activated Ca²⁺ (CRAC) channel, resulting in the influx of high concentration of calcium from outside the cell into the cell. In the original SOCE, STIM1 is anchored to the membrane of the endoplasmic reticulum (ER) and upon sensing calcium depletion of the endoplasmic reticulum, it rapidly oligomerizes and interacts with CRAC at the plasma membrane, causing extracellular calcium to enter the cytoplasm through the CRAC channel. On the other hand, in the case of OptoSTIM1, the luminal region of STIM1, the calcium-sensing EF-hand, and the luminal region of the transmembrane domain are replaced with a synthetic protein construct in which EGFP is fused to the N-terminus of the human codon-optimized PHR domain of Cryptochrome 2 (Cry2) derived from Arabidopsis thaliana. The PHR domain binds to form oligomers when exposed to blue light up to 488 nm, and upon optical stimulation, OptoSTIM1 oligomerizes and consequently activates endogenous CRAC channel. MonSTIM1 is a modified protein with increased sensitivity to light compared to OptoSTIM1 by introducing an E281A mutation and additional C-terminal 9-amino acids into the PHR domain of OptoSTIM1. Therefore, using OptoSTIM1 and monSTIM1, calcium inside cells can be increased noninvasively by irradiating blue light.

Pluripotent stem cells (PSCs), a general term for stem cells that can differentiate into virtually all cell types that make up the endoderm, mesoderm, and ectoderm, can be a platform for maximizing the usefulness of monSTIM1, which functions as a regulator of calcium, the signaling molecule. By introducing monSTIM1 into pluripotent stem cells, it can be used to investigate the role of calcium transients or trigger specific cellular mechanisms by increased intracellular calcium concentration in various types of cells. In particular, the locus at which an exogenous gene is introduced into pluripotent stem cells strongly affects the expression stability of the gene of interest, and the exogenous gene integrated into the AAVS1 genomic locus located in the first intron of PPP1R12C on chromosome 19 was confirmed to be stably and consistently expressed in both hPSCs and cells differentiated from hPSCs.

Among the cellular mechanisms in which calcium acts as a key regulator, the extracellular export of secretory molecules is one of the fastest events in response to upregulation of intracellular calcium. Insulin secretion in pancreatic endocrine β-cells is also directly related to intracellular calcium. The pancreatic islet, an endocrine microorgan of the pancreatic parenchyma, is composed of several types of endocrine cells, including α-cells that produce glucagon, β-cells that produce insulin, δ-cells that produce somatostatin, γ-cells that produce small amounts of pancreatic polypeptides (i.e., PP cells) and ε-cells that produce ghrelin. In humans, 75% of the cells that make up the pancreatic islet are β-cells, 35-40% are α-cells, and 10-15% are δ-cells. Among these, β-cells are especially the center of attention because insulin deficiency caused by β-cell failure or cell death is directly related to diabetes, a life-threatening disease worldwide. Accordingly, there is a literature disclosing that a pancreatic islet-like endocrine cell organoid can be produced from hPSCs as a method for treating diabetes (Sci Rep, 6, 35145.). Endocrine cell cluster (ECC), or pancreatic islet-like organoid (PIO), is a three-dimensional cell cluster of endocrine cells derived from hPSCs.

In pancreatic β-cells, glucose-stimulated insulin secretion (GSIS) is the primary mechanism for regulating blood glucose levels. A rapid increase in intracellular calcium in pancreatic β-cells causes exocytosis of insulin granules. Insulin vesicles are located on the plasma membrane, and when cytoplasmic calcium is detected by synaptotagmin in the vesicle membrane, the two membranes fuse and insulin molecules are secreted out of the cell. Therefore, insulin secretion can be regulated by intracellular calcium control through monSTIM1, with the potential to improve diabetes pathophysiology by regulating glycemic homeostasis in patients.

Accordingly, the present inventors selected monSTIM1 as an inducer of Ca²⁺ concentration increase (Ca²⁺ transient) specialized for spatiotemporal regulation, and used the CRISPR-Cas9 system to differentiate human pluripotent stem cells with monSTIM1 knocked-in into the AAVS1 locus of hPSCs (monSTIM1-hPSCs) into pancreatic islet-like organoids (PIOs), and then completed the present invention by manufacturing an islet-like organoid capable of optical control of insulin secretion.

### SUMMARY OF THE INVENTION

It is an object of the present invention to provide an islet-like organoid in which insulin secretion is regulated by light irradiation for use in the treatment of diabetic patients, and a preparation method of the same.

To achieve the above object, the present invention provides an islet-like organoid expressing monSTIM1 in which insulin secretion is regulated by light irradiation.

The present invention also provides a preparation method of the islet-like organoid expressing monSTIM1 comprising the following steps:
1) a step of introducing monSTIM1 into stem cells;
2) a step of differentiating the monSTIM1-introduced stem cells of step 1) to include definitive endoderm (DE) cells;
3) a step of differentiating the definitive endoderm cells of step 2) into pancreatic endoderm (PE) cells;
4) a step of differentiating the pancreatic endoderm cells of step 3) into endocrine progenitor (EP) cells;
5) a step of differentiating the endocrine progenitor cells of step 4) into hormone-expressing endocrine cells (EC); and
6) a step of differentiating the hormone-expressing endocrine cells of step 5) into an islet-like organoid (pancreatic islet-like organoid).

In addition, the present invention provides an islet-like organoid expressing monSTIM1 prepared by the preparation method.

### Advantageous Effect

The present invention relates to an islet-like organoid in which insulin secretion can be optically regulated by differentiating, into pancreatic islets, human pluripotent stem cells (monSTIM1-hPSC) obtained by knocking-in monSTIM1, which is an inducer of Ca²⁺ concentration increase (Ca²⁺ transient), into the AAVS1 locus of the stem cells. Since it has been confirmed in vitro and in the body of a diabetic mouse model that the islet-like organoid of the present invention can secrete insulin through an intracellular calcium influx increased by light irradiation, the islet-like organoid can be effectively used in the treatment of diabetic patients.

### BRIEF DESCRIPTION OF THE DRAWINGS

FIG. 1A is a schematic diagram showing the AAVS1-CAG-monSTIM1 donor plasmid for introducing monSTIM1 into the AAVS1 locus of H1hESCs, the AAVS1 locus of H1hESCs with monSTIM1 introduced at the target locus (targeted allele), and the AAVS1 locus of wild-type H1hESCs without monSTIM1 introduced. The donor plasmid contains a polynucleotide sequence encoding the monSTIM1 protein, in which nine amino acids (ARDPPDLDN, SEQ. ID. NO: 43) and a linker ([SGGGGGGG]₃) (SEQ. ID. NO: 44) are introduced into the C-terminus of the PHR domain of optoSTIM1, and glutamic acid (E), the 281st amino acid of the PHR domain, is replaced by alanine (A) (E281A).

FIG. 1B is a set of photographs showing the results of PCR performed with each primer set to confirm genotyping after introducing the donor plasmid. It was confirmed that the donor plasmid was introduced in all lanes except lane 1 (F1/R1), homozygosity (monSTIM1^{+/+}-H1) was confirmed in lane 2, heterozygosity (monSTIM1^{+/+}-H1) was confirmed in the remaining lanes (F2/R2), and the original monSTIM1 construct was introduced in all except lane 1 (F3/R3).

FIG. 1C is a set of photographs showing that the monSTIM1-knocked-in H1hESCs also expressed pluripotency markers like wild-type H1hESCs, confirmed by alkaline phosphatase staining and immunofluorescence staining.

FIG. 1D is a set of photographs confirming the morphology and GFP expression status of homozygous (monSTIM1^{+/+}-H1) and heterozygous (monSTIM1^{+/+}-H1) monSTIM1-H1-hESCs. It was confirmed that homozygous (monSTIM1^{+/+}-H1) showed stronger GFP expression than heterozygous (monSTIM1^{+/+}-H1).

FIG. 2A is a set of graphs and photographs showing that endogenous calcium ([Ca²⁺]ᵢ) was increased by blue light irradiation in homozygous (monSTIM1^{+/+}-H1) monSTIM1-H1-hESCs and that the increase in endogenous calcium ([Ca²⁺]ᵢ) was attenuated in response to treatment with SKF96365, a CRAC inhibitor (The y-axis of the bottom graph is the Ca²⁺ dye X-rhodamine fluorescence signal at each time point normalized to the fluorescence signal at t=0. Left panel of graph: untreated group, Right panel of graph: CRAC inhibitor treated group).

FIG. 2B is a set of graphs and photographs showing that endogenous calcium ([Ca²⁺]ᵢ) was increased by blue light irradiation in heterozygous (monSTIM1^{+/+}-H1) monSTIM1-H1-hESCs and that the increase in endogenous calcium ([Ca²⁺]ᵢ) was attenuated in response to treatment with SKF96365, a CRAC inhibitor (The y-axis of the bottom graph is the Ca²⁺ dye X-rhodamine fluorescence signal at each time point normalized to the fluorescence signal at t=0. Left panel of graph: untreated group, Right panel of graph: CRAC inhibitor treated group).

FIG. 2C is a set of graphs showing the difference in intracellular calcium influx in homozygous (monSTIM1^{+/+}-H1) monSTIM1-H1-hESCs when stimulated continuously for 36 seconds or when stimulated with 1 second of stimulation followed by 11 seconds of rest, and confirming that the difference in intracellular calcium influx was not significant in either case (The y-axis of the graph is the Ca²⁺ dye X-rhodamine fluorescence signal at each time point normalized to the fluorescence signal at t=0.).

FIG. 2D is a set of graphs showing the difference in intracellular calcium influx in homozygous (monSTIM1^{+/+}-H1) monSTIM1-H1-hESCs when stimulated continuously for 60 seconds or when stimulated with 1 second of stimulation followed by 11 seconds of rest, and confirming that the difference in intracellular calcium influx was not significant in either case (The y-axis of the graph is the Ca²⁺ dye X-rhodamine fluorescence signal at each time point normalized to the fluorescence signal at t=0.).

FIG. 2E is a set of graphs confirming whether the reversibility of the increase in intracellular calcium influx induced by blue light irradiation was maintained in monSTIM1-H1-hESCs and that monSTIM1^{+/+}-H1 cells exhibited repeated increases and decreases in intracellular calcium influx with blue light ON and OFF signaling compared to control H1 cells (The y-axis of the graph is the Ca²⁺ dye X-rhodamine fluorescence signal at each time point normalized to the fluorescence signal at t=0.).

FIG. 3A is a schematic diagram showing the protocol for differentiating monSTIM1-H1-hESCs and control group cells (H1-hESCs) into islet-like organoids (pancreatic islet-like organoids, PIOs).

FIG. 3B is a graph showing that the definitive endoderm markers such as SOX17, GATA4, FOXA2, and CXCR4 were expressed at similar levels in the definitive endoderm cells differentiated from monSTIM1-H1-hESCs and the definitive endoderm differentiated from control cells (H1-hESCs) (The y-axis of the graph is a value normalized to the expression level of GAPDH, a housekeeping gene.).

FIG. 3C is a graph showing that the pancreatic endoderm (PE) markers such as PDX1 and HNF1β were expressed at similar levels in the PE cells differentiated from monSTIM1-H1-hESCs and the PE cells differentiated from control cells (H1-hESCs) (The y-axis of the graph is a value normalized to the expression level of GAPDH, a housekeeping gene.).

FIG. 3D is a graph showing that the endocrine progenitor cell (EP) markers such as NKX2.2 and NGN3 were expressed at similar levels in the EP cells differentiated from monSTIM1-H1-hESCs and the EP cells differentiated from control cells (H1-hESCs) (The y-axis of the graph is a value normalized to the expression level of GAPDH, a housekeeping gene.).

FIG. 3E is a graph showing that the endocrine cell (EC) markers such as PDX1, NKX6.1, and MAFA were expressed at similar levels in the ECs differentiated from monSTIM1-H1-hESCs and the ECs differentiated from control cells (H1-hESCs) (The y-axis of the graph is a value normalized to the expression level of GAPDH, a housekeeping gene.).

FIG. 3F is a set of graphs showing that the endocrine hormones such as insulin (INS), pancreatic peptide (PPY) and somatostatin (SST), the endocrine function-related markers such as glucokinase (GCK) and glucose transporter 1 (SLC2A1), and the EC markers such as PDX1, NKX6.1 and MAFA were expressed at similar levels in the organoids differentiated from monSTIM1-H1-hESCs and the organoids differentiated from control cells (H1-hESCs) (The y-axis of the graph is a value normalized to the expression level of GAPDH, a housekeeping gene.).

FIG. 3G is a graph showing the mRNA expression levels of the endogenous CRAC component Orail at each differentiation stage, confirming that the mRNA expression levels of Orail were relatively higher in islet-like organoids (PIOs) than in ESC-stage cells, and that the expression trend of Orail according to differentiation stage showed a similar pattern between the two lines (The y-axis of the graph represents the mRNA expression level of Orail at each stage normalized to the expression level of GAPDH.).

FIG. 4A is a set of photographs showing the expressions of the markers specific to each differentiation stage, wherein the definitive endoderm cells differentiated from monSTIM1-H1-hESCs expressed FOXA2 and GATA4 at the same levels as the definitive endoderm cells differentiated from control cells (H1-hESCs), the endocrine progenitor cells differentiated from monSTIM1-H1-hESCs expressed PDX1 and NKX2.2 at the same levels as the endocrine progenitor cells differentiated from control cells (H1-hESCs), and the endocrine cells differentiated from monSTIM1-H1-hESCs expressed insulin (INS) and PDX1 at the same levels as the endocrine cells differentiated from control cells (H1-hESCs).

FIG. 4B is a set of photographs showing that the hormone-expressing islet-like organoids differentiated from monSTIM1-H1-hESCs expressed insulin (INS) and PDX1 at the same levels as the islet-like organoids differentiated from control cells (H1-hESCs), and that the islet-like organoids differentiated from monSTIM1-H1-hESCs expressed somatostatin (SST), glucagon (GCG), and pancreatic peptide (PP) at the same levels as the islet-like organoids differentiated from control cells (H1-hESCs).

FIGS. 5A and 5B are graphs showing the intracellular Ca²⁺ oscillation patterns observed in the islet-like organoids differentiated from control cells and monSTIM1^{+/+}-H1 after high concentration glucose stimulation, confirming that both the islet-like organoids differentiated from control cells and monSTIM1^{+/+}-H1 containing functional β-cells were unresponsive under basal glucose conditions and became responsive after high concentration glucose treatment (The y-axis of the graph is the Ca²⁺ dye X-rhodamine fluorescence signal at each time point normalized to the fluorescence signal at t=0.).

FIG. 6A is a set of graphs showing the intracellular calcium influx induced by light irradiation in beta cells within the organoids after applying blue light stimulation for a certain period of 12, 36, and 60 seconds to the islet-like organoids (PIOs) differentiated from control H1-hESCs or monSTIM1^{+/+}-H1 (The y-axis of the graph is a value of increase in the fluorescence signal (F) at each time point relative to the Ca²⁺ dye X-rhodamine fluorescence signal (F₀) at t=0 normalized with the value of increase in the maximum value of the observed fluorescence signal (Fₘₐₓ) over the entire imaging period compared to the fluorescence signal (F₀) at t=0.).

FIG. 6B is a series of graphs temporally continuing from FIG. 6A, showing that the light-stimulated cells were treated with 27.5 mM of high glucose, quantified, and only beta cells responding to high glucose were selected as imaging quantification targets.

FIG. 7A is a graph showing whether the islet-like organoids differentiated from monSTIM1-H1-hESCs have reversibility of intracellular calcium influx by blue light irradiation.

FIG. 7B is a graph showing the results of imaging beta cells within each group of FIG. 7A, observing calcium influx within beta cells in the organoid treated with 27.5 mM of high glucose.

FIG. 8A is a set of graphs showing that the organoids differentiated from monSTIM1^{+/+}-H1hESCs exhibited a significant increase in insulin secretion when cells were irradiated with light for 1 hour regardless of glucose concentration (8.33% duty cycle, continuous) (light+/glucose−: p<0.01, light+/glucose+: p<0.05) or when stimulated with high concentrations of glucose alone (light−/glucose+: p<0.05), compared to the organoids differentiated from control cells (H1-hESCs) that significantly secrete insulin only when stimulated with high concentrations of glucose (light-/glucose+ and light+/glucose+: p<0.05, light+/glucose−: p=0.0527).

FIG. 8B is a graph showing the results of repeating light stimulation with an 8.33% duty cycle and a duration of 1 minute at intervals of 9 minutes (p<0.05), 19 minutes (p<0.01), and 29 minutes (p<0.01) for a stimulation time of 1 hour, confirming that insulin secretion was similar to the high glucose stimulation control group (p<0.05) despite the spaced light stimulation.

FIG. 8C is a set of graphs showing the possibility of inducing repetitive and reversible insulin secretion by repeating light stimulation with an 8.33% duty cycle and a duration of 1 minute at 9-minute intervals and at 6-hour, 12-hour (p<0.05), or 24-hour (p<0.05) intervals.

FIG. 9A is a set of photographs showing that the donor plasmid was introduced in all lanes except lane 1 (F1/R1), homozygosity (monSTIM1^{+/+}-ND-iPSC) was confirmed in lane 2, heterozygosity (monSTIM1^{+/+}-ND-iPSC) was confirmed in the remaining lanes (F2/R2), and the original monSTIM1 construct was introduced in all except lane 1 (F3/R3).

FIG. 9B is a set of photographs confirming whether monSTIM1-ND-iPSCs expressed pluripotency markers.

FIG. 9C is a set of graphs confirming that the heterozygous gene mutation of KCNJ11 was preserved in monSTIM1-ND-iPSCs (In the sequence represented by SEQ. ID. NO: 45, the 11^{th} nucleotide was substituted from G to A.).

FIG. 9D is a set of graphs and photographs confirming that the monSTIM1 expressed in ND-iPSCs increased endogenous calcium ([Ca²⁺]ᵢ) by blue light irradiation, and attenuated the increase in endogenous calcium ([Ca²⁺]ᵢ) in both cell lines in response to treatment with SKF96365, a CRAC inhibitor.

FIG. 9E is a set of photographs confirming that the monSTIM1-transfected ND-iPSCs (monSTIM1^{+/+}-ND-iPSCs) strongly expressed GFP.

FIG. 9F is a set of graphs showing the repetitive increases and decreases in intracellular calcium influx along with blue light ON and OFF signals in monSTIM1^{+/+}-ND-iPSCs, compared to control ND cells.

FIG. 10A is a set of photographs confirming that the definitive endoderm cells differentiated from monSTIM1-ND-iPSCs expressed FOXA2 and GATA4 at the same levels as the definitive endoderm cells differentiated from control group cells (ND-iPSCs), the pancreatic endoderm cells differentiated from monSTIM1-ND-iPSCs expressed HNF4α at the same level as the pancreatic endoderm cells differentiated from control group cells (ND-iPSCs), the endocrine progenitor cells differentiated from monSTIM1-ND-iPSCs expressed PDX1 and NKX2.2 at the same levels as the endocrine progenitor cells differentiated from control group cells (ND-iPSCs), and the hormone-expressing endocrine cells differentiated from monSTIM1-ND-iPSCs expressed PDX1 and insulin (INS) at the same levels as the hormone-expressing endocrine cells differentiated from control group cells (ND-iPSCs).

FIG. 10B is a set of photographs confirming that the islet-like organoid differentiated from monSTIM1-ND-iPSCs expressed insulin (INS) and PDX1 at the same levels as the islet-like organoid differentiated from control group cells (ND-iPSCs), and the islet-like organoid differentiated from monSTIM1-ND-iPSCs expressed somatostatin (SST) and pancreatic peptide (PP) at the same levels as the islet-like organoid differentiated from control group cells (ND-iPSCs).

FIG. 10C is a set of graphs confirming that the insulin secretion did not occur when high concentrations of glucose stimulation alone was applied to the islet-like organoids differentiated from control ND-iPSCs and monSTIM1-ND-iPSCs, or when light stimulation was applied to the ND-iPSC-derived islet-like organoid, but that the light-induced insulin secretion occurred when light stimulation was applied to the islet-like organoid derived from monSTIM1-ND-iPSCs (p<0.05).

FIG. 11A is a set of a graph and photographs showing the insulin secretion capacity of monSTIM1^{+/+}-PIOs encapsulated in low and high density PCL sheets (left) and PCL sheets induced by in vitro light stimulation (Relative insulin levels are expressed as the fold change between the insulin levels secreted before and after stimulation.) (mean±SEM, n=3).

FIG. 11B is a set of photographs showing the mouse before and after surgical implantation of an encapsulated PIO implant.

FIG. 11C is a set of a photograph and a graph showing the monSTIM1^{+/+}-PIO implant encapsulated in a PCL sheet recovered after transplantation (left) and the level of human c-peptide in the serum of a mouse implanted with the monSTIM1^{+/+}-PIO induced by light stimulation (n=7 (for intraperitoneal glucose infusion), n=8 (for LED cage)).

FIG. 11D is a photograph showing the expression of insulin and monSTIM1 in the monSTIM1^{+/+}-PIO implant encapsulated with a PCL sheet recovered after implantation.

FIG. 12 is a diagram showing the mechanism by which insulin secretion is regulated by intracellular calcium control through monSTIM1.

### DESCRIPTION OF THE PREFERRED EMBODIMENTS

Hereinafter, the present invention is described in detail.

The present invention provides an islet-like organoid expressing monSTIM1 in which insulin secretion is regulated by light irradiation.

The light may be blue light with a wavelength of 470 to 500 nm, and preferably can be blue light of 488 nm.

The monSTIM1 is activated by light irradiation and increases intracellular Ca²⁺ influx.

The intracellular calcium influx can be reversibly regulated by light irradiation.

Insulin secretion is promoted by the increase of the intracellular calcium influx.

The light irradiation may be continuous or in a cycle of 8.33%, in which light is irradiated for 1 second and light is not irradiated for 11 seconds.

The light irradiation may be performed at intervals of 9 to 29 minutes in a cycle of irradiating light for 1 second and not irradiating light for 11 seconds.

The islet-like organoid includes beta cells (0-cells) that cause an increase in intracellular calcium influx upon light irradiation.

The present invention also provides a preparation method of the islet-like organoid expressing monSTIM1 comprising the following steps:
1) a step of introducing monSTIM1 into stem cells;
2) a step of differentiating the monSTIM1-introduced stem cells of step 1) to include definitive endoderm (DE) cells;
3) a step of differentiating the definitive endoderm cells of step 2) into pancreatic endoderm (PE) cells;
4) a step of differentiating the pancreatic endoderm cells of step 3) into endocrine progenitor (EP) cells;
5) a step of differentiating the endocrine progenitor cells of step 4) into hormone-expressing endocrine cells (EC); and
6) a step of differentiating the hormone-expressing endocrine cells of step 5) into an islet-like organoid (pancreatic islet-like organoid).

The stem cells may be embryonic stem cells, induced pluripotent stem cells, or adult stem cells, and may be of human origin, but not always limited thereto.

The induced pluripotent stem cells may be generated from dermal fibroblasts of a neonatal diabetes patient, but not always limited thereto.

The monSTIM1 can be introduced into the AAVS1 gene locus located in the first intron of PPP1R12C on chromosome 19 of the stem cell.

The monSTIM1 may comprise a nucleotide sequence represented by SEQ. ID. NO: 46.

The stem cells into which the monSTIM1 has been introduced may be homozygous clones or heterozygous clones, and preferably may be homozygous clones.

In the differentiation of steps 2) to 6), stem cells are differentiated in a Matrigel-coated culture vessel to create an environment similar to the cell microenvironment.

The culture of step 2) is performed in a medium containing Activin A, CHIR9902, and LiCl or a medium containing Activin A.

The Activin A can be included in the medium at 30 to 70 ng/ml, preferably 40 to 60 ng/ml, and more preferably 45 to 55 ng/ml.

The CHIR99021 can be included in the medium at 1 to 5 μM, preferably 2 to 4 μM, and more preferably 2.5 to 3.5 μM.

The LiCl can be included in the medium at 0.5 to 3.5 mM, preferably 1 to 3 mM, and more preferably 1.5 to 2.5 mM.

The differentiation of step 2) may be performed for 2 to 6 days, preferably for 3 to 5 days.

The definitive endoderm cells may be contained in 90 to 98% of the total cells, specifically 92 to 96%, and more specifically 93 to 95% of the total cells.

The definitive endoderm cells can express SOX17, GATA4, FOXA2 or CXCR4.

The culture of step 3) is performed in a medium containing retinoic acid, dorsomorphin, SB431542, FGF2, and SANT1.

The retinoic acid may be included in the medium at 1 to 3 μM, preferably 1.5 to 2.5 μM.

The dorsomorphin may be included in the medium at 1 to 3 μM, preferably 1.5 to 2.5 μM.

The SB431542 may be included in the medium at 5 to 15 μM, preferably 7 to 13 μM, and more preferably 8 to 12 μM.

The FGF2 may be included in the medium at 1 to 9 ng/ml, preferably 3 to 7 ng/ml, and more preferably 4 to 6 ng/ml.

The SANT1 may be included in the medium at 100 to 400 nM, preferably 150 to 350 nM, and more preferably 200 to 300 nM.

The differentiation of step 3) may be performed for 2 to 10 days, preferably for 3 to 9 days, and more preferably for 4 to 8 days.

The pancreatic endoderm cells may express PDX1 or HNF1β.

The culture of step 4) is performed in a medium containing ascorbic acid, dorsomorphin, SB431542, and DAPT.

The ascorbic acid may be included in the medium at 30 to 70 μg/ml, preferably 40 to 60 μg/ml, and more preferably 45 to 55 μg/ml.

The dorsomorphin may be included in the medium at 1 to 3 μM, preferably 1.5 to 2.5 μM.

The SB431542 may be included in the medium at 5 to 15 μM, preferably 7 to 13 μM, and more preferably 8 to 12 μM.

The DAPT may be included in the medium at 5 to 15 μM, preferably 7 to 13 μM, and more preferably 8 to 12 μM.

The differentiation of step 4) may be performed for 2 to 6 days, preferably for 3 to 5 days.

The endocrine progenitor cells may express NKX2.2 or NGN3.

The culture of step 5) is performed in a medium containing glucose, dibutyryl-cAMP, dorsomorphin, exendin-4, SB431542, SB431542, nicotinamide, and ascorbic acid.

The glucose may be included in the medium at 10 to 40 mM, preferably to 30 mM.

The dibutyryl-cAMP may be included in the medium at 100 to 900 μM, preferably 300 to 700 μM, and more preferably 400 to 600 μM.

The exendin-4 may be included in the medium at 5 to 15 μM, preferably 7 to 13 μM, and more preferably 8 to 12 μM.

The dorsomorphin may be included in the medium at 1 to 3 μM, preferably 1.5 to 2.5 μM.

The SB431542 may be included in the medium at 5 to 15 μM, preferably 7 to 13 μM, and more preferably 8 to 12 μM.

The nicotinamide may be included in the medium at 5 to 15 mM, preferably 7 to 13 mM, and more preferably 8 to 12 mM.

The ascorbic acid may be included in the medium at 30 to 70 μg/ml, preferably 40 to 60 μg/ml, and more preferably 45 to 55 μg/ml.

The differentiation of step 5) may be performed for 2 to 14 days, preferably for 5 to 11 days, and more preferably for 6 to 10 days

The hormone-expressing endocrine cells may express PDX1, SST, INS or PPY.

The culture of step 6) is performed in a medium containing glucose, dibutyryl-cAMP, dorsomorphin, exendin-4, SB431542, dorsomorphin, SB431542, nicotinamide, and ascorbic acid.

The glucose may be included in the medium at 5 to 45 mM, preferably to 40 mM, and more preferably 20 to 30 mM

The dibutyryl-cAMP may be included in the medium at 100 to 900 μM, preferably 300 to 700 μM, and more preferably 400 to 600 μM.

The exendin-4 may be included in the medium at 5 to 15 μM, preferably 7 to 13 μM, and more preferably 8 to 12 μM.

The dorsomorphin may be included in the medium at 1 to 3 μM, preferably 1.5 to 2.5 μM.

The SB431542 may be included in the medium at 5 to 15 μM, preferably 7 to 13 μM, and more preferably 8 to 12 μM.

The nicotinamide may be included in the medium at 5 to 15 mM, preferably 7 to 13 mM, and more preferably 8 to 12 mM.

The ascorbic acid may be included in the medium at 30 to 70 μg/ml, preferably 40 to 60 μg/ml, and more preferably 45 to 55 μg/ml.

The differentiation of step 6) may be performed for 1 to 5 days, preferably for 1 to 4 days, and more preferably for 1 to 3 days

The hormone-expressing endocrine cells may express PDX1, SST, INS or PPY.

In addition, the present invention provides an islet-like organoid expressing monSTIM1 prepared by the preparation method.

The islet-like organoid expressing monSTIM1 can be encapsulated in a porous support and implanted, preferably encapsulated in polycaprolactone.

The islet-like organoid can be applied for the treatment of diabetes.

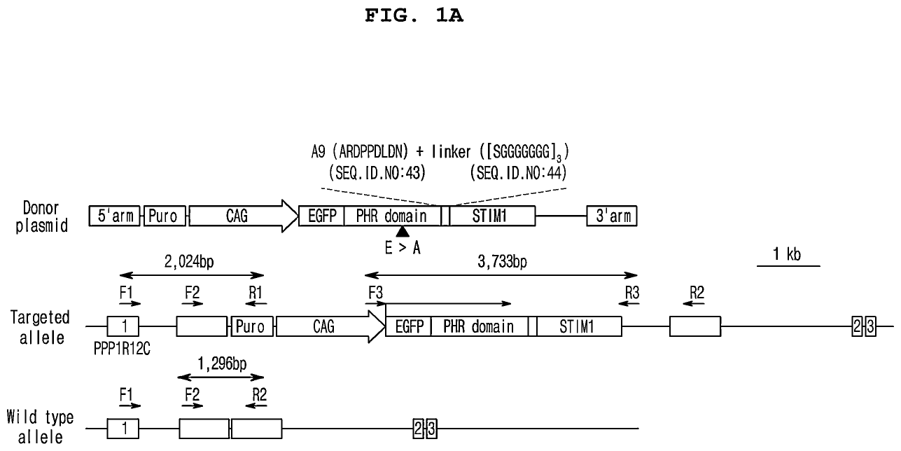

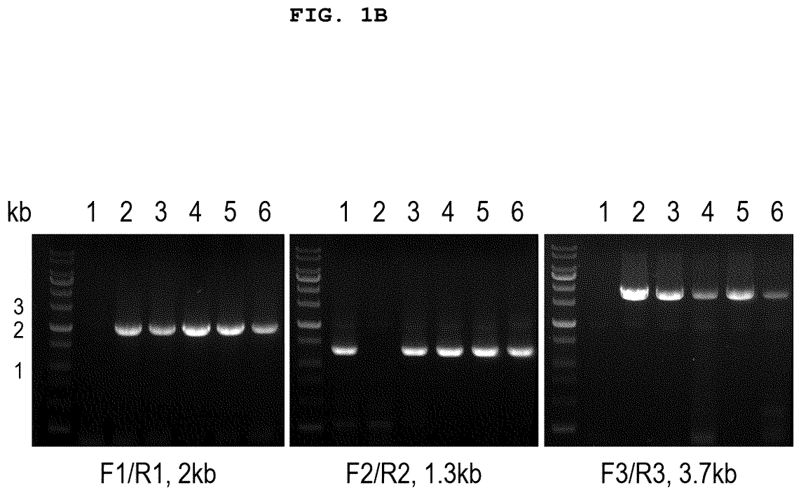

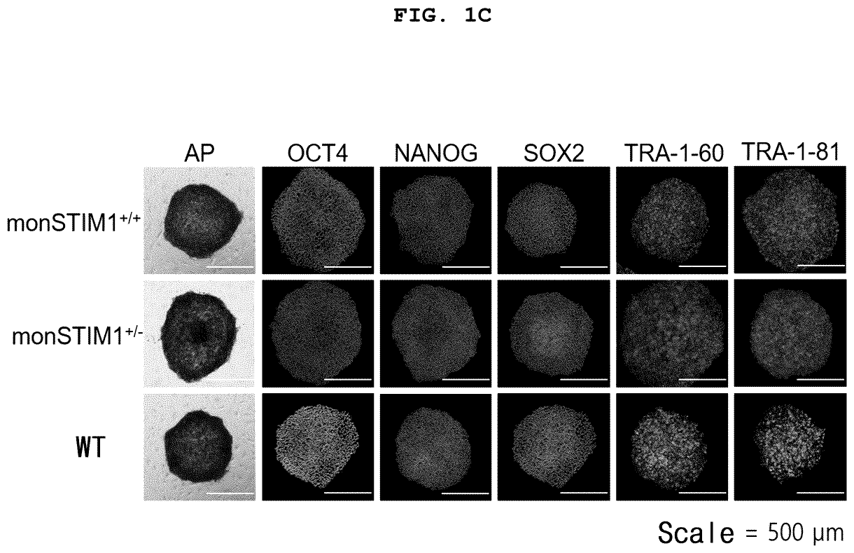

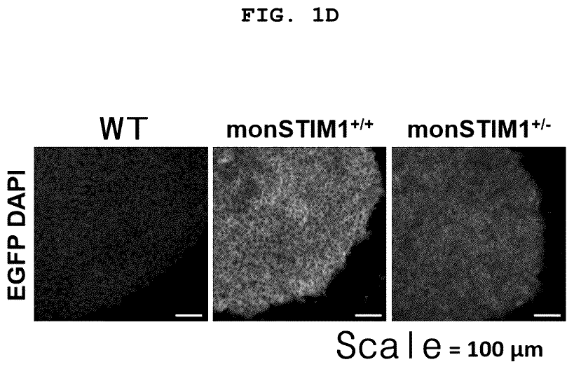

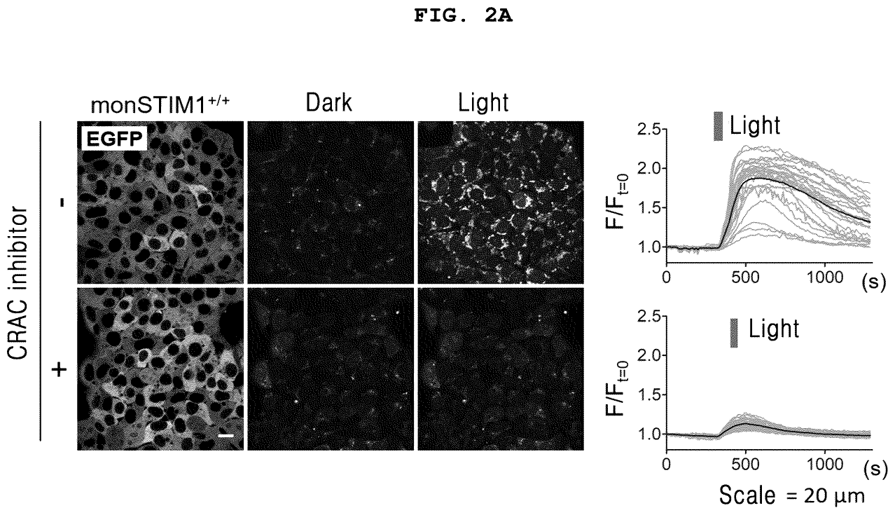

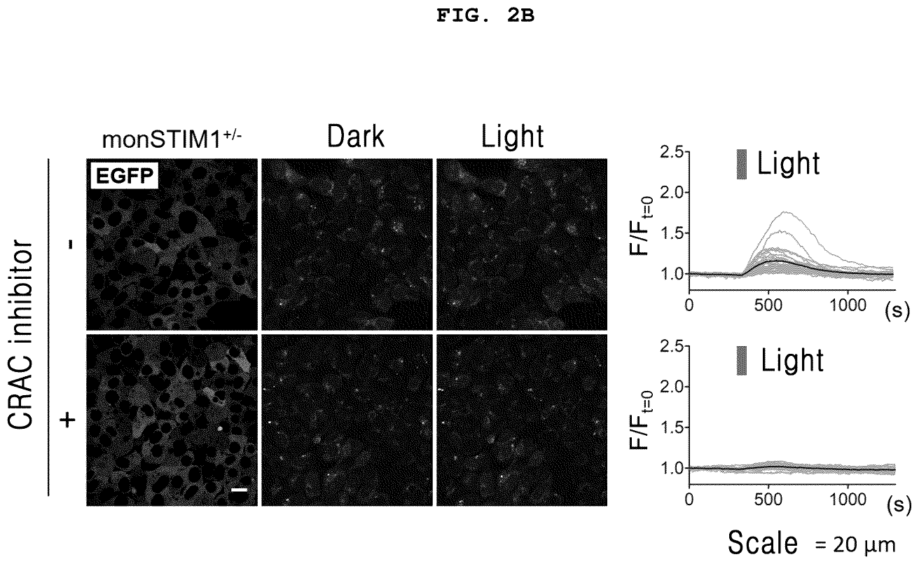

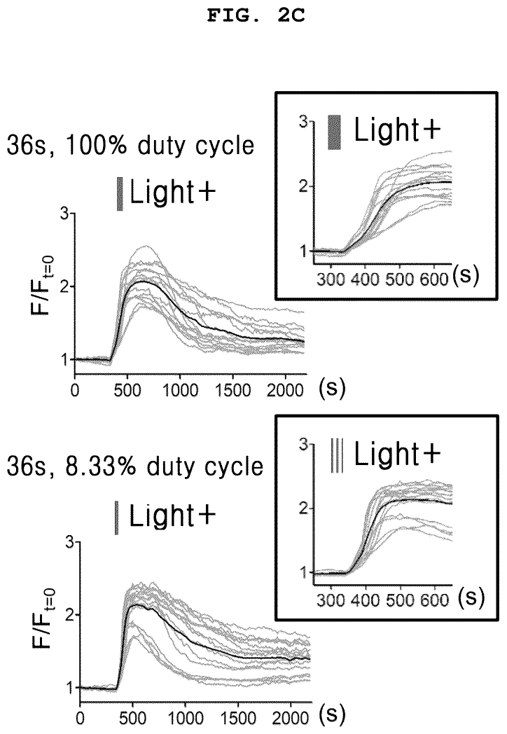

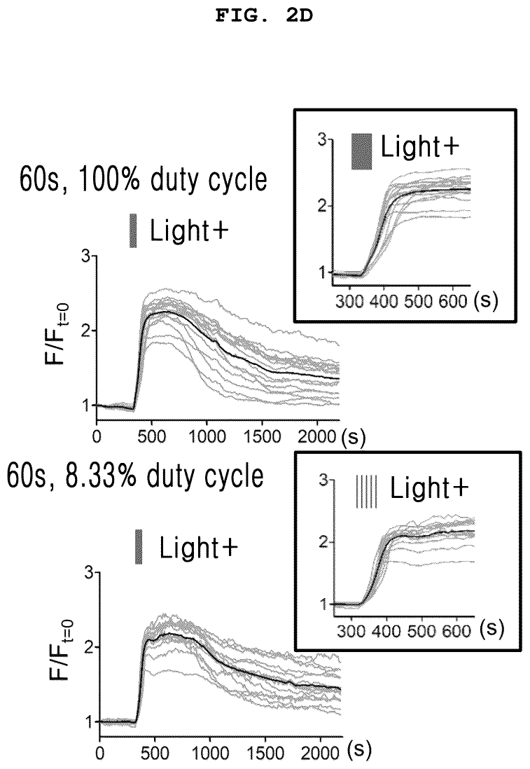

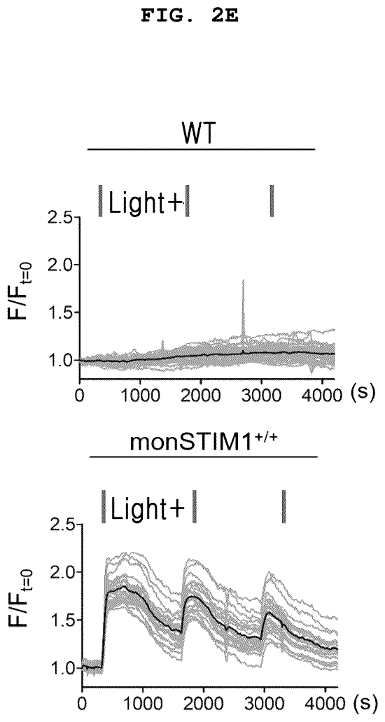

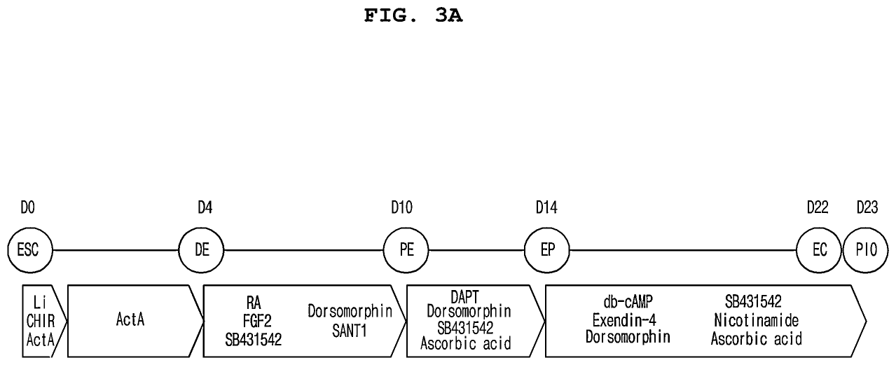

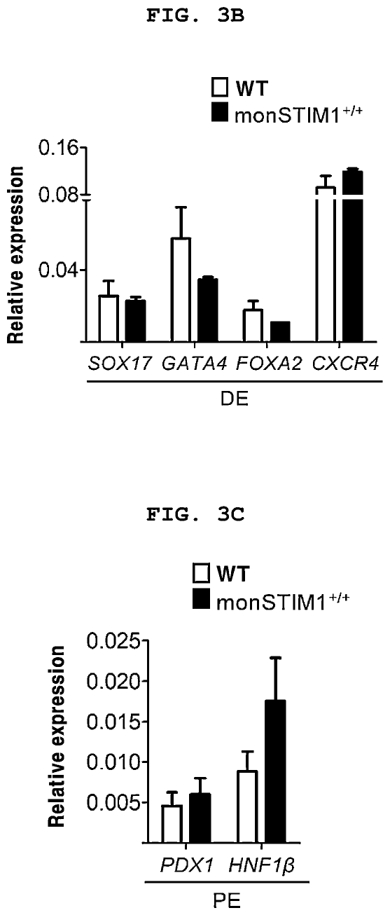

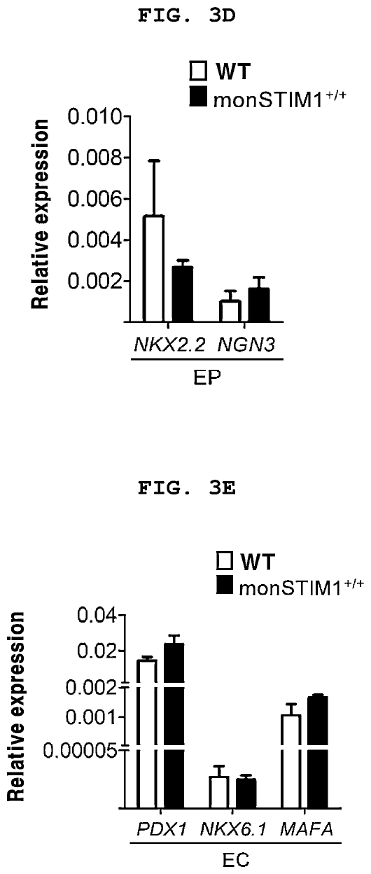

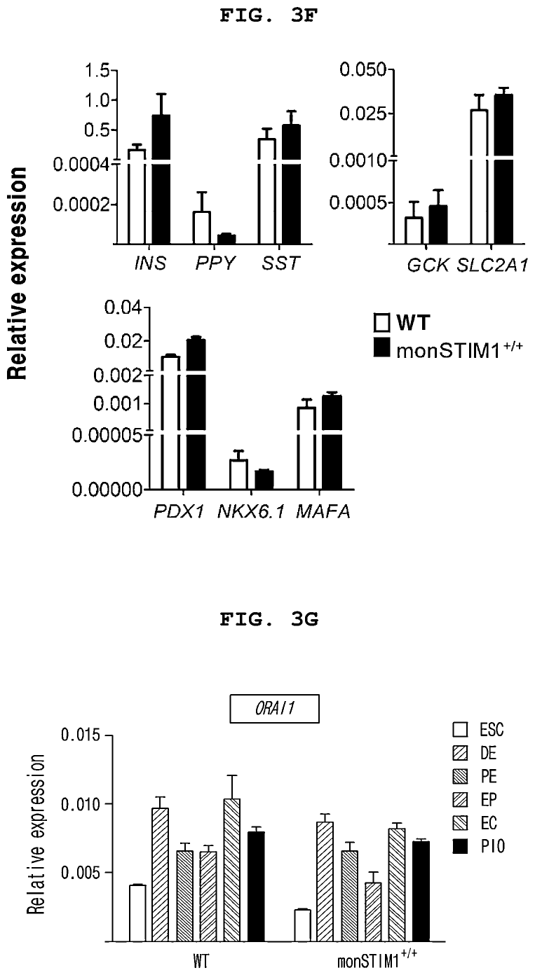

In a preferred embodiment of the present invention, monSTIM1-embryonic stem cells (monSTIM1-H1-hESCs) were prepared by knocking in the monSTIM1 vector into the AAVS1 gene locus of H1 human embryonic stem cells using the CRISPR-Cas9 system (see FIGS. 1A and 1B), and the pluripotency of monSTIM1-H1-hESCs was confirmed (see FIGS. 1C and 1D). It was confirmed that the monSTIM1 increased endogenous calcium ([Ca²⁺]ᵢ) by blue light irradiation, which decreased in response to treatment with SKF96365, a CRAC inhibitor (see FIGS. 2A and 2B), and that this was a reversible reaction by demonstrating repeated increases and decreases in intracellular calcium influx with blue light ON and OFF signaling (see FIG. 2E). In addition, monSTIM1-H1-hESCs were differentiated from definitive endoderm cells, pancreatic endoderm cells, endocrine progenitor cells, and hormone-expressing endocrine cells into islet-like organoids (see FIGS. 3A to 3F), and the mRNA expression levels of CRAC were measured at each stage.

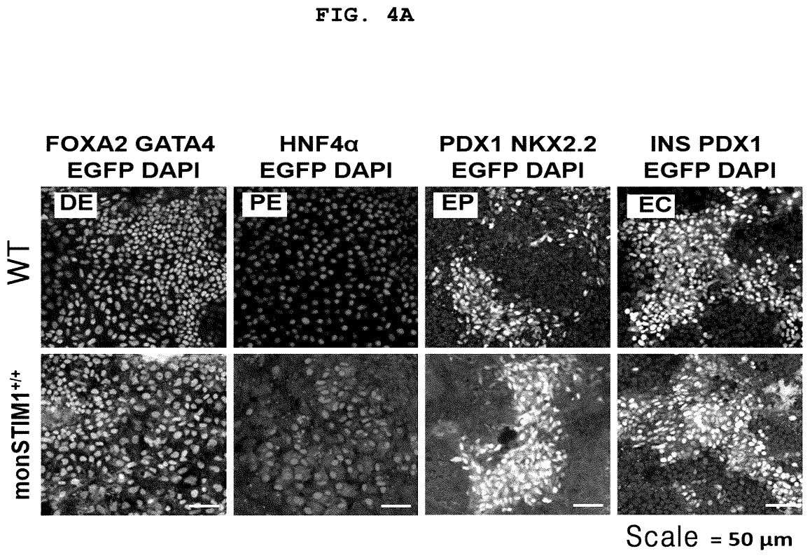

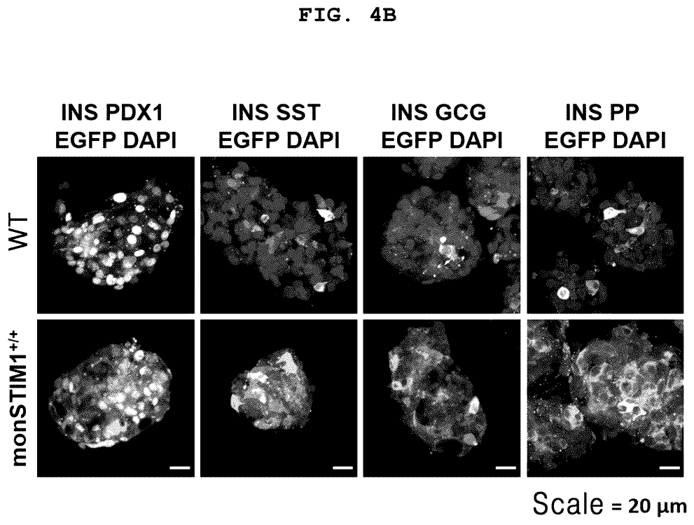

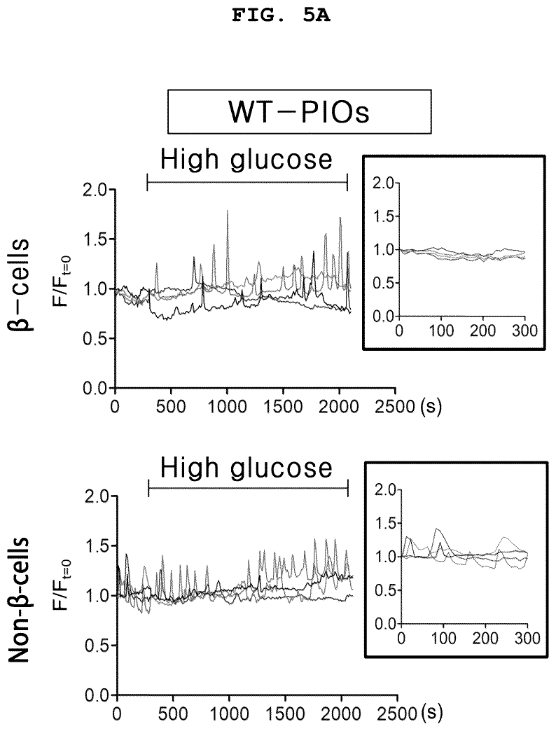

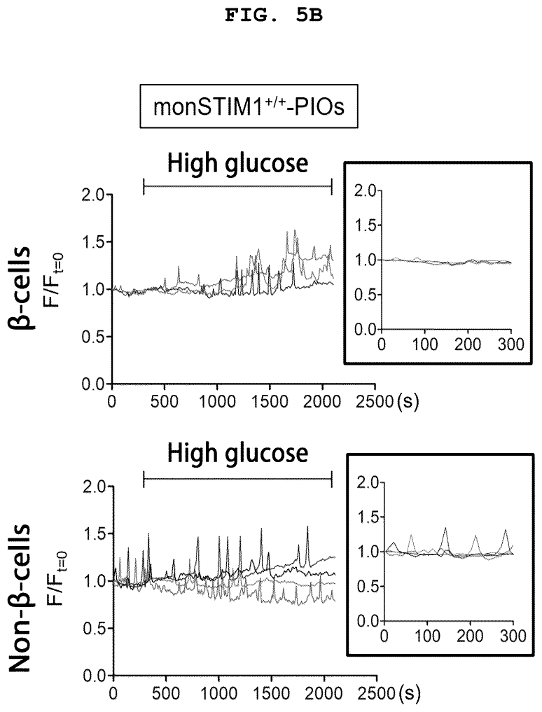

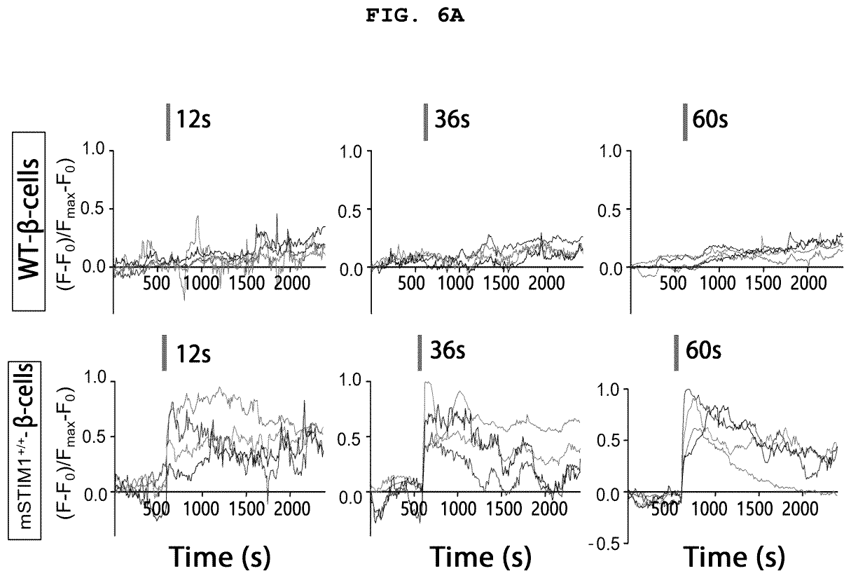

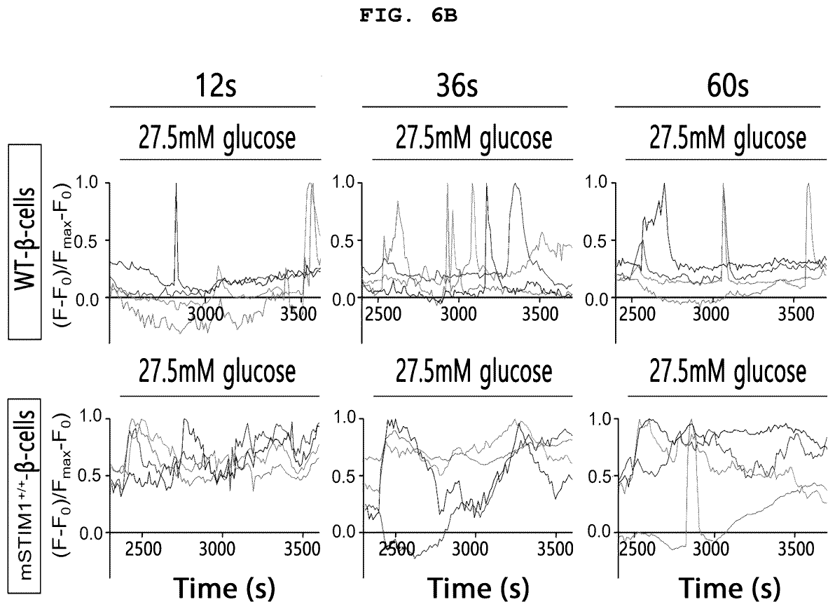

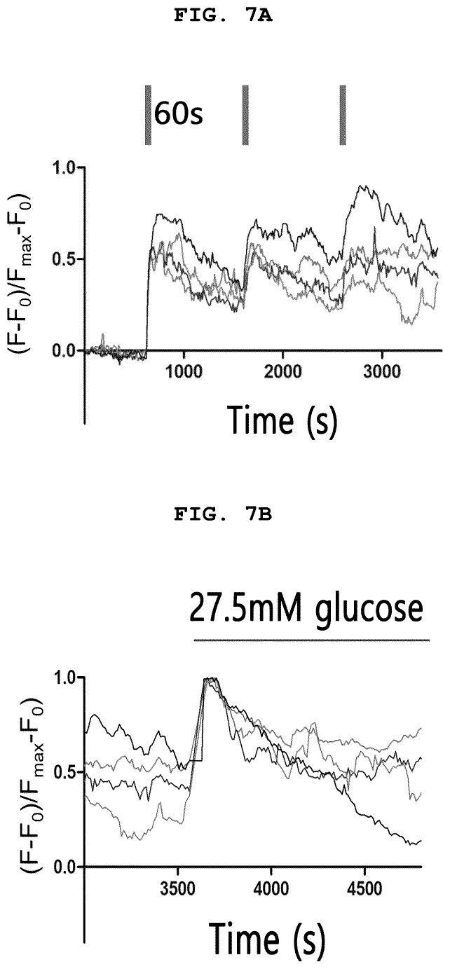

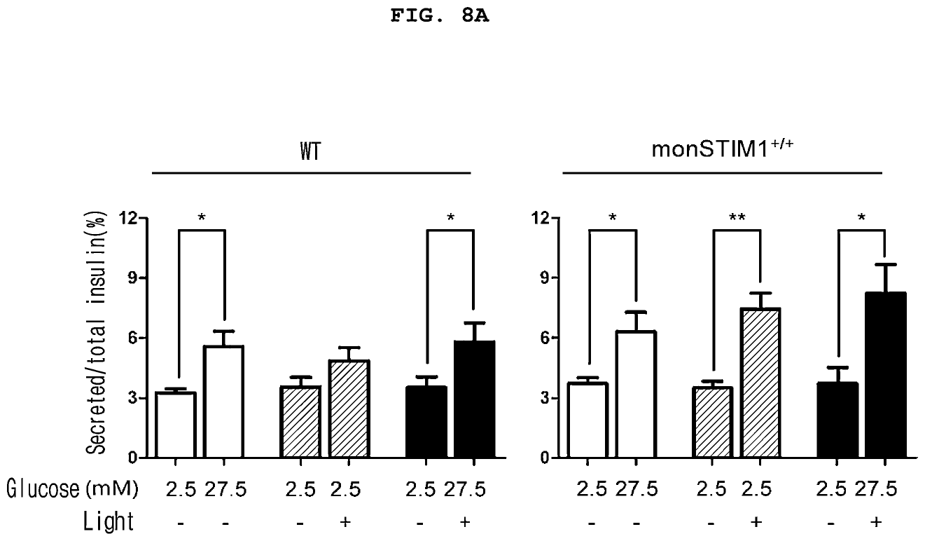

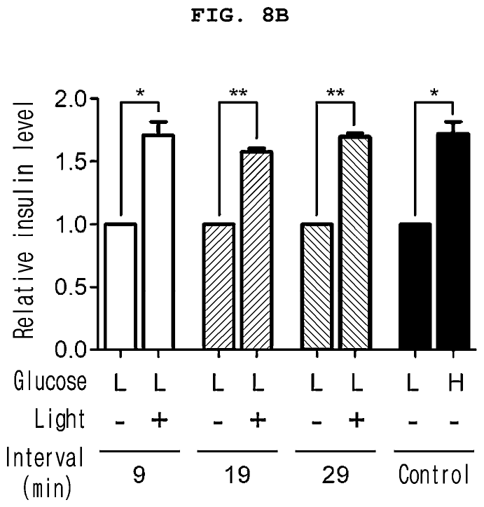

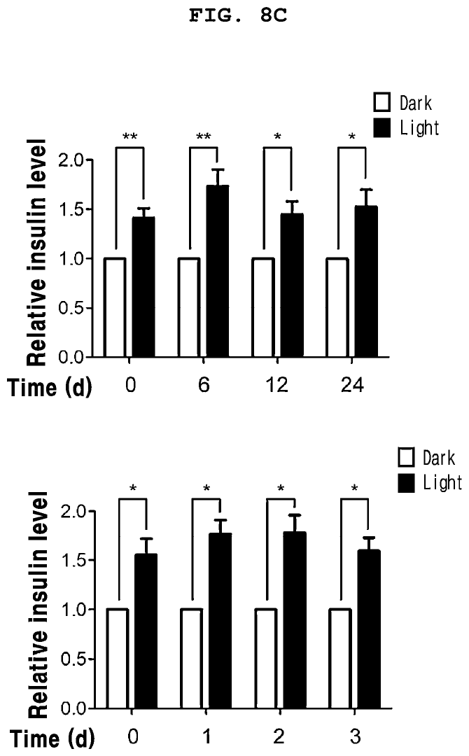

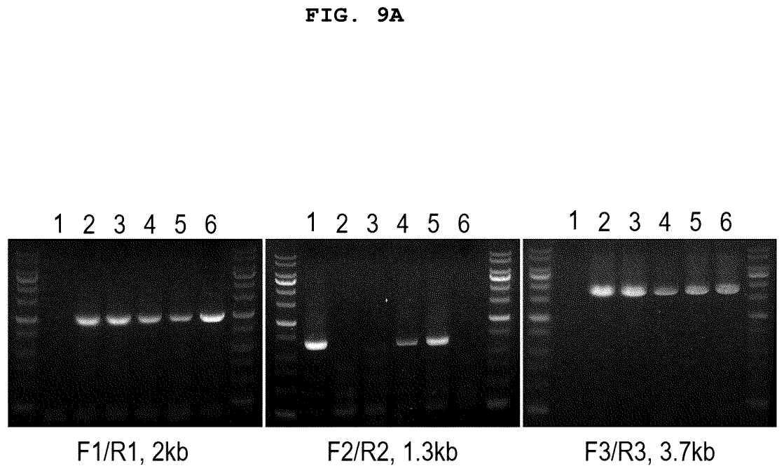

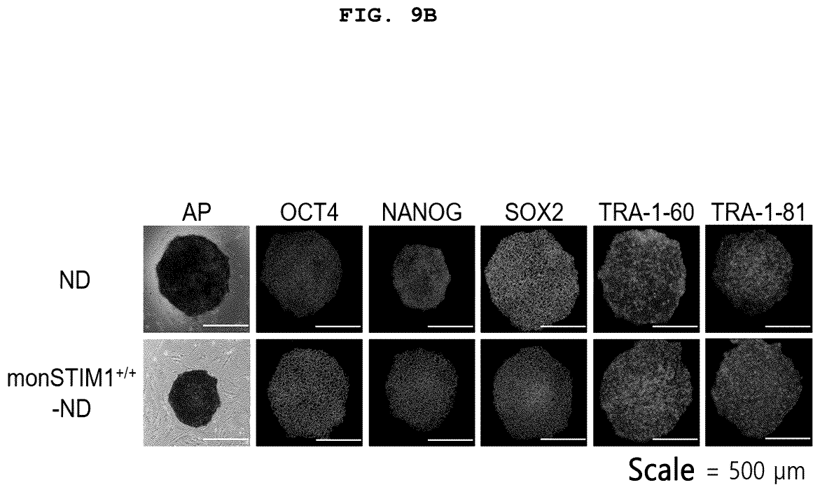

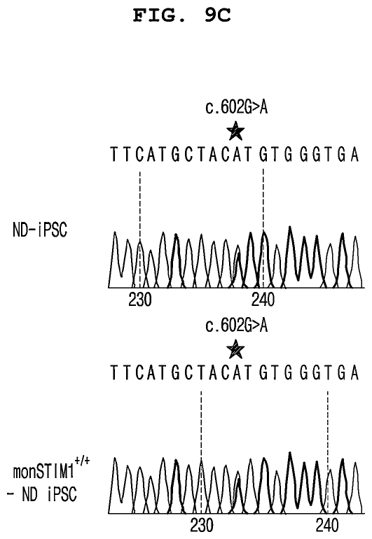

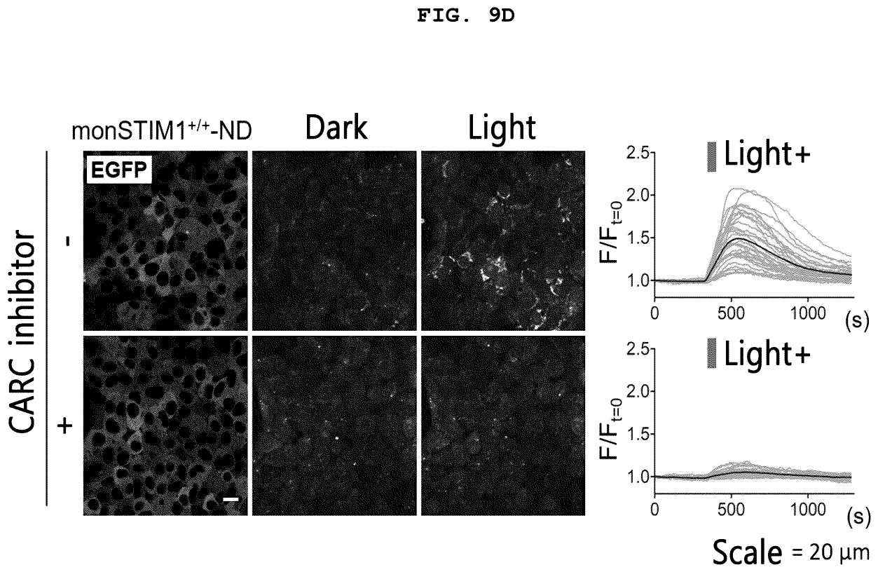

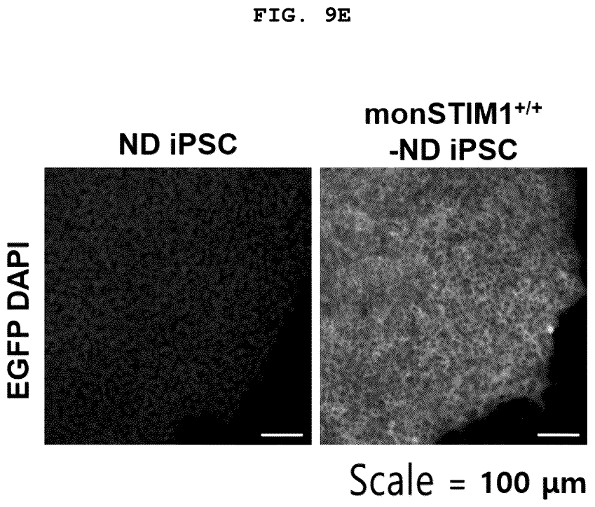

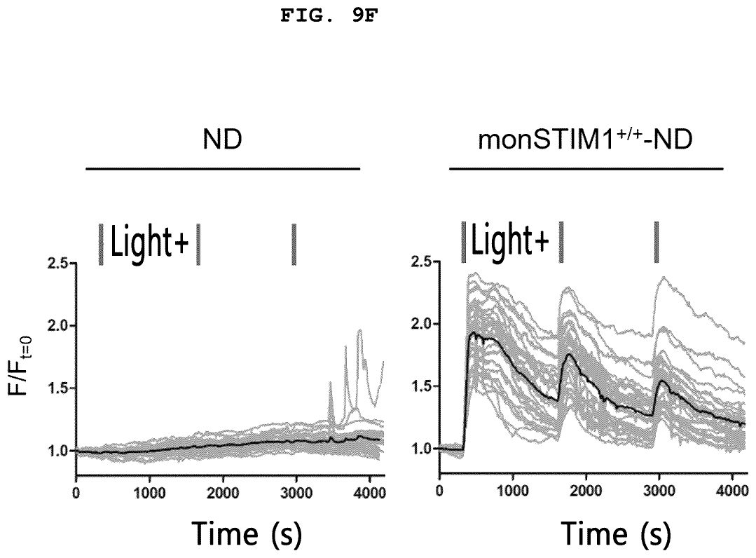

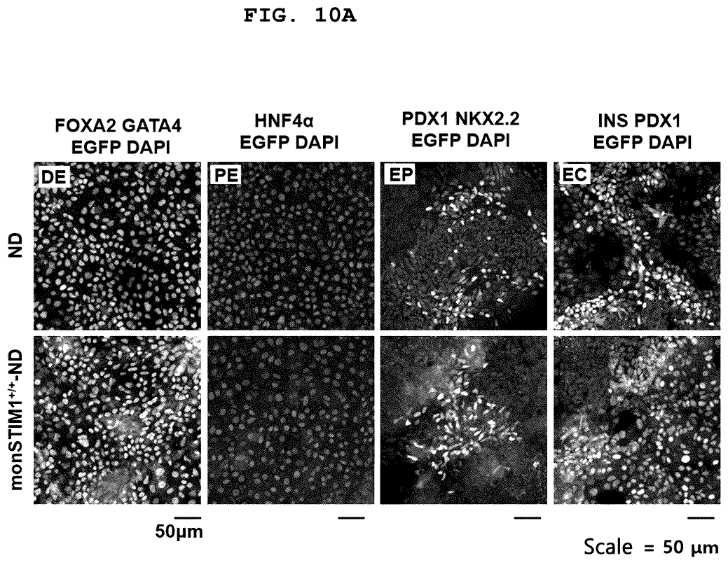

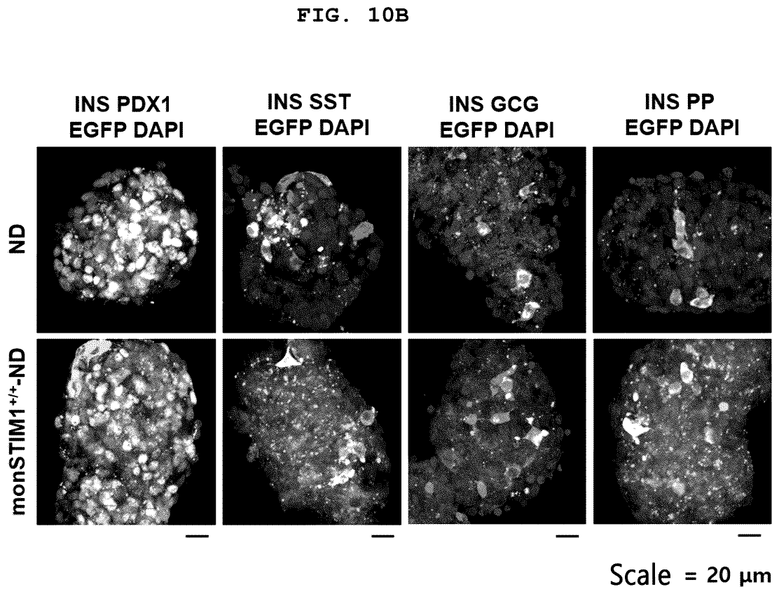

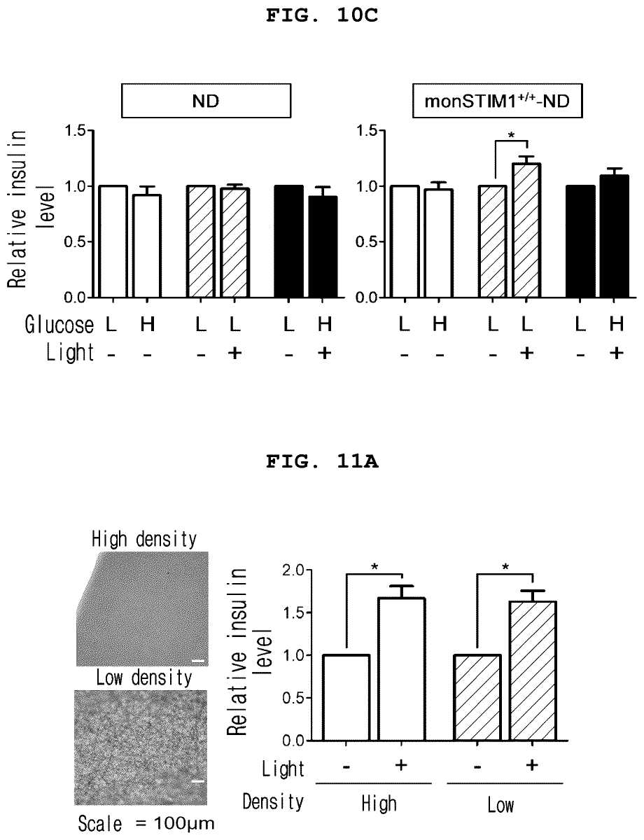

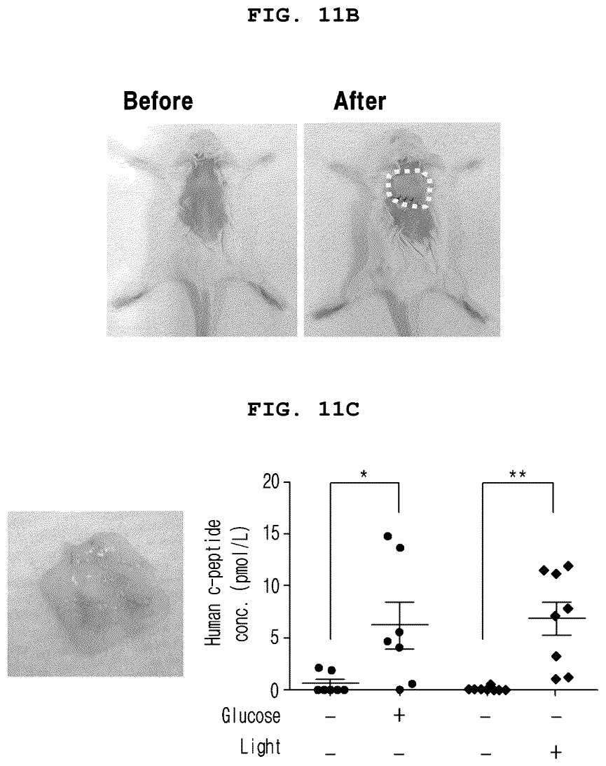

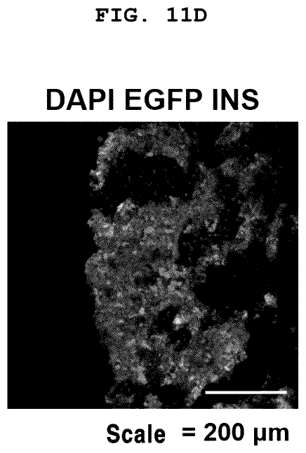

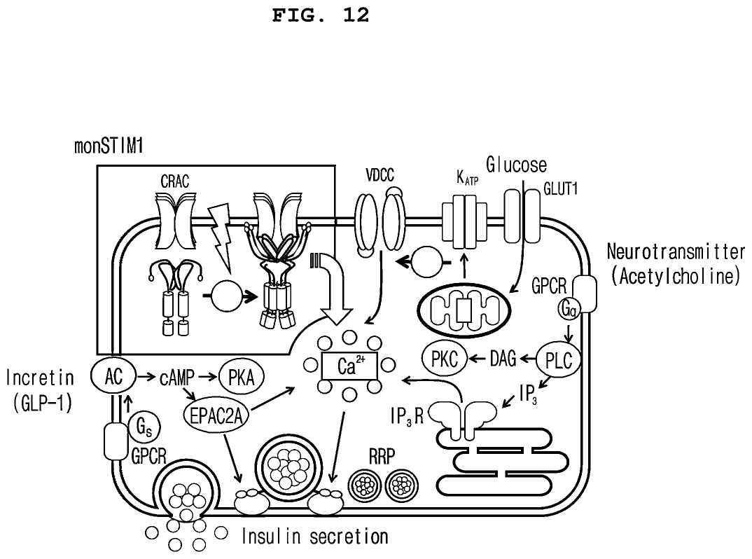

As a result, it was confirmed that the islet-like organoid cells were suitable for light-activated monSTIM1 (see FIG. 3G). In addition, it was confirmed that the islet-like organoid differentiated from monSTIM1-H1-hESCs expressed the markers specific to each stage of differentiation at the same level as the islet-like organoid differentiated from control cells (H1-hESCs) (see FIGS. 4A and 4B). It was also confirmed that Ca²⁺ oscillation occurred by glucose stimulation in the islet-like organoid prepared by differentiating monSTIM1-H1-hESCs (see FIGS. 5A and 5B), and that intracellular calcium influx occurred by light irradiation (see FIG. 6A), which could be reversibly controlled by repeated light irradiation (see FIGS. 7A and 7B). In addition, it was confirmed that the islet-like organoid differentiated from monSTIM1^{+/+}-H1hESCs secreted insulin by continuous light irradiation regardless of glucose concentration (see FIG. 8A), and secreted insulin repeatedly by multiple light inductions (see FIG. 8C). Furthermore, the present inventors prepared monSTIM1-ND-iPSCs by introducing monSTIM1 into the AAVS1 locus of the induced pluripotent stem cells prepared from dermal fibroblasts of neonatal diabetes patients (see FIG. 9A), and confirmed that the monSTIM1-ND-iPSCs expressed pluripotency markers (see FIG. 9B). It was confirmed that the monSTIM1-ND-iPSCs preserved genetic mutations specific to neonatal diabetes patients (see FIG. 9C), that endogenous calcium ([Ca²⁺ ]ᵢ) was increased in the monSTIM1-ND-iPSCs by blue light irradiation (see FIGS. 9D and 9E), and that the increased intracellular calcium influx was reversible (see FIG. 9F). The present inventors confirmed that the islet-like organoid was produced from monSTIM1-ND-iPSCs, and that the markers specific to each differentiation stage were expressed (see FIGS. 10A and 10B). It was also confirmed that applying light stimulation to the islet-like organoid differentiated from monSTIM1-ND-iPSCs could cause insulin secretion (see FIG. 10C). Finally, the present inventors confirmed in vitro that the monSTIM1^{+/+}-PIO encapsulated in a PCL sheet increased insulin secretion by light stimulation (see FIG. 11A), and transplanted the monSTIM1^{+/+}-PIO into a diabetic mouse model (see FIG. 11B). Then, it was confirmed that human c-peptide was secreted not only by glucose but also by light stimulation (see FIG. 11C), and that monSTIM1 and insulin were expressed in the monSTIM1^{+/+}-PIO recovered after transplantation (see FIG. 11D).

Therefore, the islet-like organoid expressing monSTIM1 in which insulin secretion is regulated by light irradiation of the present invention is an established model of cell therapy for treating diabetes and can be effectively used in the treatment of diabetes.

Hereinafter, the present invention will be described in detail by the following examples.

However, the following examples are only for illustrating the present invention, and the contents of the present invention are not limited thereto.

### Example 1: Construction of monSTIM1-Knockin H1-Human Embryonic Stem Cell Line (H1-hESC)

MonSTIM1-embryonic stem cells (monSTIM1-H1-hESCs) were prepared by knocking-in the monSTIM1 vector into the AAVS1 gene locus of H1 human embryonic stem cells using the CRISPR-Cas9 system as follows.

### <1-1> Construction of Expression Plasmid

An AAVS1-CAG-monSTIM1 donor plasmid was constructed using a Cas9 expression plasmid (pCas9_GFP), an AAVS1 targeting gRNA (gRNA_AAVS1-T2), an AAVS1 targeting homology arm for homologous recombination (AAVS1-CAG-hrGFP), and a monSTIM1 expression plasmid (pCMV-monSTIM1).

Specifically, hrGFP cDNA of AAVS1-CAG-hrGFP was replaced with the PCR-amplified monSTM1 cDNA sequence using the In-Fusion Cloning Kit (Clontech, Takara Bio USA, Inc., California, USA). The AAVS1-CAG-hrGFP vector was cleaved by treatment with Sa1I and M1uI for 2 hours at 37° C., and electrophoresis was performed on a 1% agarose gel for 30 hours. To remove the hrGFP structure, gel purification was performed using a DNA purification kit (Cosmo Genetech, Seoul, Korea). PCR amplification of monSTIM1 cDNA was performed using CloneAmp HiFi PCR Premix (Clontech) according to the manufacturer's instructions, using the DNA oligomers 5′-AAAGAATTCGTCGACATGGTGAGCAAGGGCGAG-3′ (SEQ. ID. NO: 1) and 5′-AGTGAATTCACGCGTCGGTGGATCCCAATTCCTAC-3′ (SEQ. ID. NO: 2) as forward and reverse primers, with the pCMV-monSTIM1 plasmid as a template. The reaction conditions were as follows; predenaturation at 98° C. for 3 minutes, denaturation at 98° C. for 10 seconds, annealing at 55° C. for seconds, polymerization at 72° C. for 4 minutes, 40 cycles from denaturation to polymerization, and final extension at 72° C. for 5 minutes.

The constructed AAVS1-CAG-monSTIM1 donor plasmid was clonally amplified in LB culture medium (LPS solution) prior to plasmid preparation, extracted using NucleoBondXtra Maxi Plus kit (Macherey-Nagel, Pennsylvania, USA), and transformed into Stellar™ Competent Cells (Clontech).

As a result, as shown in FIG. 1A, a donor plasmid containing the coding sequence of monSTIM1 and an AAVS1 homologous arm homology recombination cassette was constructed.

<1-2> Construction of monSTIM1-H1-hESC

The donor plasmid constructed in Example <1-1> above was introduced into H1-ESCs to prepare monSTIM1-H1-hESCs.

Specifically, H1-hESCs were co-transfected with a donor plasmid, a Cas9 expression plasmid, and a gRNA plasmid targeting the AAVS1 locus via electroporation using the NEON transfection system. H1-hESCs were dissociated into single cells using Accutase, and 1.25×10⁶ cells were resuspended in 100 μl of pre-warmed R buffer. The resuspended cells were then mixed with 5 μg of DNA (1.25 μg of Cas9, 1.25 μg of gRNA, and 2.5 μg of donor plasmid) and electroporated at 1400 V, 20 ms, 2 pulses according to the manufacturer's instructions. The electroporated cells were resuspended in mTeSR medium supplemented with 10 μM Y-27632 and plated at a density of 1.25×10⁶ cells/well in Matrigel-coated 4-well plates. Then, the medium was changed daily and the cells were cultured for 5 days with or without division until the cells grew to ˜50% density. For antibiotic selection, the cells were separated into single cells by treating with 0.5 μg/ml puromycin in the culture medium for 10 days, and serially diluted in a 96-well plate for clone isolation. Single colonies were generated in the plated wells at a minimum density of 10 cells/well. To select more homozygous knockin clones, the colonies selected based on hPSC morphology and relative GFP expression levels were mechanically separated from the 96-well plate, transferred to 4-well plates, and amplified for further analysis.

<1-3> Confirmation of monSTIM1-H1-hESC Generation

To confirm whether monSTIM1 was knocked-in at the AAVS1 gene locus of H1-hESCs, polymerase chain reaction (PCR) was performed as follows.

Particularly, the zygosity of monSTIM1 knocked-in at the AAVS1 gene locus of H1-hESCs was confirmed using primer set 1 (F1/R1) to detect the genomic region from the exon of PPP1R12C to the puromycin region of the homologous recombination cassette to determine whether the donor plasmid has been introduced, primer set 2 (F2/R2) to confirm the zygosity (homozygous/heterozygous) of each clone, and primer set 3 (F3/R3) to confirm if the original monSTIM1 construct has been introduced (Table 1).

Genomic DNA was extracted from each colony using the G-DEX Genomic DNA extraction kit according to the manufacturer's instructions. PCR was performed using a Taq polymerase as follows; predenaturation at 95° C. for 2 minutes, denaturation at 95° C. for 20 seconds, annealing at 60° C. for seconds, polymerization at 72° C. for 20 seconds, 35 cycles from denaturation to polymerization, and final extension at 72° C. for 5 minutes.

As a result, as shown in FIG. 1B, it was confirmed that the donor plasmid was introduced in all lanes except lane 1 (F1/R1), homozygosity (monSTIM1^{+/+}-H1) was confirmed in lane 2, heterozygosity (monSTIM1^{+/-}-H1) was confirmed in the remaining lanes (F2/R2), and the original monSTIM1 construct was introduced in all except lane 1 (F3/R3). Clones in lane 2 showing homozygosity (monSTIM1^{+/+}-H1), and lane 6 showing heterozygosity (monSTIM1^{+/-}-H1) were selected and used in the following experiments.

TABLE 1

| Primer | Sequence (5→3) | SEQ. ID. NO |
|---|---|---|
| F1 | ACCAACGCCGACGGTATCAG | SEQ. ID. NO: 3 |
| R1 | CAGACCCTTGCCCTGGTGGT | SEQ. ID. NO: 4 |
| F2 | CTTTCTCTGACCTGCATTCT | SEQ. ID. NO: 5 |
| R2 | CTACTGGCCTTATCTCACAG | SEQ. ID. NO: 6 |
| F3 | AAAGAATTCGTCGACATGGTGAGCA | SEQ. ID. NO: 7 |
| | AGGGCGAG | |
| R3 | AGTGAATTCACGCGTCGGTGGATCC | SEQ. ID. NO: 8 |
| | CAATTCCTAC | |

<1-4> Confirmation of Pluripotency of monSTIM1-H1-hESC

Alkaline phosphatase staining was performed using the leukocyte alkaline phosphatase kit (Sigma). Cells were fixed with a fixing solution consisting of 1 ml of citrate solution, 2.6 ml of acetone, and 320 μl of 37% formaldehyde (Sigma), and the fixed cells were washed with distilled water and then stained with alkaline phosphatase (AP) staining solution containing 100 μl of sodium nitrate, 100 μl of FBB-alkaline solution and 100 μl of naphthol As—BI alkaline solution for 1 hour.

For immunocytochemistry (ICC), the cultured cells were fixed with 4% formaldehyde for 30 minutes at room temperature or overnight at 4° C. The fixed cells were washed twice with PBS for 5 minutes each, permeabilized with PBS containing 0.5% Triton-X for 30 minutes, and treated with 0.1% Tween-20 (PBST; Tween-20) for 5 minutes each. The cells were blocked with PBST containing 1% bovine serum albumin (BSA) for 1 hour at room temperature and then cultured with 1% BSA containing primary antibodies overnight at 4° C. On another day, the cells were washed five times with PBST for 5 minutes each, and then cultured with secondary antibodies diluted in 1% BSA containing 1 μg/ml/DAPI for 1 hour at room temperature. The stained cells were washed five times with PBST for 5 minutes each and mounted on slide glasses using fluorescent mounting medium to observe GFP expression.

As a result, as shown in FIG. 1C, like wild-type H1-hESCs, the homozygous clones of lane 2 (monSTIM1^{+/+}-H1) and the heterozygous clones of lane 6 (monSTIM1^{+/-}-H1) were confirmed to express pluripotency markers such as OCT4, NANOG, SOX2, TRA-1-60 and TRA-1-81. In addition, it was confirmed that homozygous (monSTIM1^{+/+}-H1) exhibited stronger GFP expression than heterozygous (monSTIM1^{+/-}-H1), as shown in FIG. 1D.

### Example 2: Confirmation of monSTIM1 Activation by Blue Light Irradiation in monSTIM1-H1-hESCs

<2-1> Confirmation of Intracellular Calcium Influx Activation by Blue Light Irradiation in Homozygous (monSTIM1^{+/+}-H1) and Heterozygous (Monstim1^{+/+}-H1) Monstim1-H1-Hescs

In the monSTIM1-H1-hESCs prepared in Example 1, whether the monSTIM1 could induce intracellular Ca²⁺ transient upon stimulation with 488 nm blue light through CRAC, a component of the endogenous CRAC channel, was confirmed as follows.

Specifically, the homozygous (monSTIM1^{+/+}-H1) and heterozygous (monSTIM1^{+/-}-H1) monSTIM1-H1-hESCs selected in Examples <1-3> were isolated with Accutase, resuspended in mTeSR medium supplemented with 10 μM Y-27632, plated on a 24-well glass bottom plate coated with Matrigel 24 hours prior, and imaged at a density of 1.1×10⁵ cells/cm². On the day of imaging, cells were cultured with 2 μM of X-rhod-1, a red Ca²⁺ indicator, containing 0.02% Pluronic F-127 in mTeSR medium for 30 minutes at 37° C. Then, the residual dye was washed away with mTeSR and the cells were cultured for an additional 15 to 30 minutes before live cell imaging.

To examine the dependence on Ca²⁺-release-activated Ca²⁺ (CRAC) channel during stimulation of monSTIM1-H1-hESCs with blue light, the cells were pretreated with 50 μM of SKF96365, a CRAC channels inhibitor, for 90 minutes before imaging. All the live cell imaging processes, including blue light excitation of cells, were performed with a 488 nm laser module mounted on a Nikon A1 confocal microscope at a power intensity of 204.5 μW/mm². Blue light was irradiated under the conditions in Table 2 below.

As a result, as shown in FIGS. 2A and 2B, the monSTIM1 expressed in H1-hESCs increased endogenous calcium ([Ca²⁺]ᵢ) by blue light irradiation, and attenuated the increase in endogenous calcium ([Ca²⁺]ᵢ) in both cell lines in response to treatment with SKF96365, a CRAC inhibitor. In addition, the endogenous calcium ([Ca²⁺]ᵢ) level reached a maximum within 200 to 250 seconds from the start of stimulation and was deactivated to half of the maximum within 650 to 900 seconds from the start of stimulation. These results suggest that the intracellular Ca²⁺ influx induced by light irradiation is dependent on the endogenous CRAC channel. In addition, the relative fluorescence intensity (normalized to the intensity at t=0) of [Ca²⁺]ᵢ generated in the homozygous cell line (monSTIM1^{+/+}-H1) (FIG. 2A) was 1.5-fold higher at the point of maximum compared to that in the heterozygous cell line (monSTIM1^{+/-}-H1) (FIG. 2B).

TABLE 2

| FIG. | Light irradiation conditions |
|---|---|
| FIGS. 2A | Baseline imaging for 5 minutes with 561 nm laser, Imaging |
| and 2B | for 60 seconds with 488 nm/561 nm laser (8.33% duty cycle), |
| | and Resting imaging for 15 minutes with 561 nm laser |

### <2-2> Determination of Blue Light Dose

Using homozygous (monSTIM1^{+/+}-H1) monSTIM1-H1-hESCs, it was confirmed whether the low-dose pulsed light irradiation to reduce the phototoxic effect that causes oxidative stress on living cells affects the light-induced intracellular calcium influx process as follows.

Specifically, intracellular calcium influx was confirmed in the same manner as in Example <2-1> except that the homozygous (monSTIM1^{+/+}-H1) monSTIM1-H1-hESC cell line was irradiated with blue light under the conditions shown in Table 3 below.

As a result, as shown in FIGS. 2C and 2D, the difference in intracellular calcium influx observed when the cell line was stimulated with blue light for 36 or 60 seconds was not significant, and it was possible that intracellular calcium influx could be induced at lower duty cycles. Therefore, a blue light irradiation dose of 8.33% duty cycle was selected.

TABLE 3

| FIG. | Light irradiation conditions |
|---|---|
| FIG. 2C | Baseline imaging for 5 minutes with 561 nm laser, Imaging for |
| | 36 seconds with 488 nm/561 nm laser (duty cycle of FIG. 2C, 1 |
| | second ON, 11 seconds OFF), and Resting imaging for 30 |
| | minutes with 561 nm laser |
| FIG. 2D | Baseline imaging for 5 minutes with 561 nm laser, Imaging for |
| | 60 seconds with 488 nm/561 nm laser (duty cycle of FIG. 2D, 1 |
| | second ON, 11 seconds OFF), and Resting imaging for 30 |
| | minutes with 561 nm laser |

<2-3> Confirmation of Reversibility of Intracellular Calcium Influx by Blue Light Irradiation in monSTIM1-H1-hESCs

Considering previous findings that a reversible Ca²⁺ elevation can be induced by light irradiation in hESCs overexpressing OptoSTIM1 (Nat Biotechnol, 33(10), 1092-1096.), whether the reversibility of the increase in intracellular calcium influx caused by blue light irradiation was maintained in monSTIM1-H1-hESCs was confirmed as follows.

Specifically, the intracellular calcium influx was confirmed in the same manner as in Example <2-1>, except that control H1-hESCs and (monSTIM1^{+/+}-H1) monSTIM1-H1-hESCs were stimulated with 5 sets of blue light pulses repeated 3 times at 20-minute intervals (Table 4).

As a result, as shown in FIG. 2E, compared to control H1 cells, monSTIM1^{+/+}-H1 cells showed repetitive increases and decreases in intracellular calcium influx accompanied by blue light ON and OFF signals. The above results suggest that intracellular calcium can be dynamically regulated in the monSTIM1-H1-hESC cell line.

TABLE 4

| FIG. | Light irradiation conditions |
|---|---|
| FIG. 2E | Baseline imaging for 5 minutes with 561 nm laser, [Imaging |
| | for 60 seconds with 488 nm/561 nm laser (8.33% duty cycle), |
| | and Resting imaging for 20 minutes with 561 nm laser] ×3 |

### Example 3: Preparation of Islet-Like Organoids from monSTIM1-H1-hESCs

As shown in FIG. 3A, monSTIM1-H1-hESCs and control cells (H1-hESCs) were differentiated according to a previously reported differentiation protocol (Kim, Y et al. Islet-like organoids derived from human pluripotent stem cells efficiently function in the glucose responsiveness in vitro and in vivo. Sci Rep 6, 35145 (2016).) in the following order: definitive endoderm (DE) cells, pancreatic endoderm (PE) cells, endocrine progenitor (EP) cells, hormone-expressing endocrine cells (EC), and pancreatic islet-like organoids (PIO).

<3-1> Differentiation of Definitive Endoderm (DE) from monSTIM1-H1-hESCs

Prior to differentiation induction, H1-hESC or monSTIM1-H1-hESC colonies were dissociated into single cells by incubation in EDTA/PBS solution at 37° C. for 8 minutes. The isolated cells were seeded in Matrigel-coated 4-well plates at a density of 4×10⁴ cells/well and then cultured for 2 days in a feeder-free system in mTeSR medium supplemented with 10 μM Y27632.

To induce the differentiation of monSTIM1-H1-hESCs and control cells (H1-hESCs) into definitive endoderm (DE) cells, monSTIM1-H1-hESCs or H1-hESCs were cultured for 1 day in basal DMEM/F12 supplemented with 0.2% BSA, 50 ng/ml of Activin A, 3 μM CHIR99021, and 2 mM LiCl. Then, the cells were cultured for 3 days in basic DMEM/F12 supplemented with 0.2% BSA, 1% B27 supplement, and 50 ng/ml of Activin A.

Afterwards, qRT-PCR was performed to measure the mRNA expression levels of definitive endoderm markers (SOX17, GATA4, FOXA2, and CXCR4). Total RNA was extracted from cells using Easy-BLUE reagent, and 1 μg of total RNA was reverse transcribed into cDNA using the 1st-strand cDNA synthesis kit. The mixture for the qRT-PCR reaction consisted of 40 mM Tris (pH 8.4, LPS solution), 0.1 M KCl, 6 mM MgCl₂, 2 mM dNTP, 0.2% fluorescein, 0.4% SYBR Green, and 10% DMSO. Reaction and reading were performed with the CFX Connect TM Real-Time System using the primers shown in Table 5 below under the following conditions: 95° C. for 10 minutes, followed by 30 seconds at 95° C., 30 seconds at 55-60° C., 30 seconds at 72° C., and 39 cycles of plate reading and melting curve detection. The expression level of the target gene to the housekeeping gene was determined by the Ct value of the target gene normalized to the GAPDH gene.

TABLE 5

| Primer | Sequence (5→3) | SEQ. ID. NO |
|---|---|---|
| SOX17 forward | ACCAACGCCGACGGTATCAG | SEQ. ID. NO: 9 |
| primer | | |
| SOX17 reverse | GCGGCCGGTACTTGTAGTT | SEQ. ID. NO: 10 |
| primer | | |
| GATA4 forward | TCCAAACCAGAAAACGGAAG | SEQ. ID. NO: 11 |
| primer | | |
| GATA4 reverse | CTGTGCCCGTAGTGAGATGA | SEQ. ID. NO: 12 |
| primer | | |
| FOXA2 forward | AACAAGATGCTGACGCTGAG | SEQ. ID. NO: 13 |
| primer | | |
| FOXA2 reverse | CAGGAAACAGTCGTTGAAGG | SEQ. ID. NO: 14 |
| primer | | |
| CXCR4 forward | GGTGGTCTATGTTGGCGTCT | SEQ. ID. NO: 15 |
| primer | | |
| CXCR4 reverse | TGGAGTGTGACAGCTTGGAG | SEQ. ID. NO: 16 |
| primer | | |

As a result, as shown in FIG. 3B, it was confirmed that the definitive endoderm markers such as SOX17, GATA4, FOXA2, and CXCR4 were expressed at similar levels in the definitive endoderm cells differentiated from monSTIM1-H1-hESCs and the definitive endoderm differentiated from control group cells (H1-hESCs).

<3-2> Differentiation of Pancreatic Endoderm (PE) from Definitive Endoderm (De)

The definitive endoderm cells of Example <3-1> were cultured in the stage II medium for 6 days to differentiate into pancreatic endoderm (PE) cells. The stage II medium consisted of DMEM high glucose medium supplemented with 0.5% B27 supplement, 2 μM retinoic acid (RA, Sigma), 2 μM dorsomorphin (AG Scientific), 10 μM SB431542, 5 ng/ml of FGF2 (basic fibroblast growth factor), and 250 nM SANT1. The mRNA expression levels of PE markers such as PDX1 and HNF1β were measured by performing qRT-PCR with the differentiated pancreatic endoderm (PE) using the same method and conditions as in Example <3-1> except that the primers shown in Table 6 below were used.

TABLE 6

| Primer | Sequence (5→3) | SEQ. ID. NO |
|---|---|---|
| PDX1 forward | ACCAACGCCGACGGTATCAG | SEQ. ID. NO: 17 |
| primer | | |
| PDX1 reverse | AACATAACCCGAGCACAAGG | SEQ. ID. NO: 18 |
| primer | | |
| HNF1ß forward | AGCCCACCAACAAGAAGATG | SEQ. ID. NO: 19 |
| primer | | |
| HNF1ß reverse | CATTCTGCCCTGTTGCATTC | SEQ. ID. NO: 20 |
| primer | | |

As a result, as shown in FIG. 3C, it was confirmed that the pancreatic endoderm (PE) markers such as PDX1 and HNF1β were expressed at similar levels in the PE differentiated from monSTIM1-H1-hESCs and the PE differentiated from control group cells (H1-hESCs).

<3-3> Differentiation of Endocrine Progenitor (EP) Cells from Pancreatic Endoderm (PE)

The pancreatic endoderm cells of Example <3-2> were cultured in the stage III medium for 4 days to differentiate into endocrine progenitor (EP) cells. The stage III medium consisted of DMEM supplemented with 0.5% B27 supplement, 50 μg/ml of ascorbic acid, 2 μM dorsomorphin, 10 μM SB431542, and 10 μM DAPT.

The mRNA expression levels of EP markers such as NKX2.2 and NGN3 were measured by performing qRT-PCR with the differentiated endocrine progenitor (EP) cells using the same method and conditions as in Example <3-1> except that the primers shown in Table 7 below were used.

TABLE 7

| Primer | Sequence (5→3) | SEQ. ID. NO |
|---|---|---|
| NKX2.2 forward | TGGCCATGTAAACGTTCTGA | SEQ. ID. NO: 21 |
| primer | | |
| NKX2.2 reverse | GGAAGAAAGCAGGGGAAAA | SEQ. ID. NO: 22 |
| primer | C | |
| NGN3 forward | GGCTGTGGGTGCTAAGGGTA | SEQ. ID. NO: 23 |
| primer | AG | |
| NGN3 reverse | CAGGGAGAAGCAGAAGGAA | SEQ. ID. NO: 24 |
| primer | CAA | |

As a result, as shown in FIG. 3D, it was confirmed that the endocrine progenitor (EP) cell markers such as NKX2.2 and NGN3 were expressed at similar levels in the EP differentiated from monSTIM1-H1-hESCs and the EP differentiated from control group cells (H1-hESCs).

<3-4> Differentiation of Hormone-Expressing Endocrine (EC) Cells from Endocrine Progenitor (EP) Cells

The endocrine progenitor (EP) cells of Example <3-3> were cultured in the stage IV medium for 8 days to differentiate into endocrine cells (EC). The stage IV medium consisted of CMRL 1066 supplemented with 0.5% B27 supplement, 0.5% penicillin-streptomycin, 25 mM glucose, 500 μM dibutyryl-cAMP, 10 μM exendin-4, 2 μM dorsomorphin, 10 μM SB431542, 10 mM nicotinamide, and 50 μg/ml of ascorbic acid.

The mRNA expression levels of EC markers such as PDX1, NKX6.1, and MAFA were measured by performing qRT-PCR with the differentiated endocrine cells (EC) using the same method and conditions as in Example <3-1> except that the primers shown in Table 8 below were used.

TABLE 8

| Primer | Sequence (5→3) | SEQ. ID. NO |
|---|---|---|
| NKX6.1 forward | ATTCGTTGGGGATGACA | SEQ. ID. NO: 25 |
| primer | GAG | |
| NKX6.1 reverse | TGGGATCCAGAGGCTTA | SEQ. ID. NO: 26 |
| primer | TTG | |
| MAFA forward | CTTCAGCAAGGAGGAGG | SEQ. ID. NO: 27 |
| primer | TCATC | |
| MAFA reverse | CTCGTATTTCTCCTTGT | SEQ. ID. NO: 28 |
| primer | ACAGGTCC | |

As a result, as shown in FIG. 3E, it was confirmed that the endocrine cell (EC) cell markers such as PDX1, NKX6.1, and MAFA were expressed at similar levels in the EC differentiated from monSTIM1-H1-hESCs and the EC differentiated from control group cells (H1-hESCs).

<3-5> Differentiation of Pancreatic Islet-Like Organoids from Hormone-Expressing Endocrine Cells (EC)

For cluster formation, hormone-expressing endocrine cells (EC) were dissociated into single cells by treatment with Accutase for 15 minutes at 37° C. To increase cell viability, cells were treated with 10 μM Y27632, and the isolated ECs were placed in each well of an uncoated 96-well plate (5×10⁴ cells/well) and cultured in the stage IV medium of Example <3-4> for 1 day at 37° C., 5% Co₂.

The mRNA expression levels of endocrine hormones such as insulin (INS), pancreatic peptide (PPY) and somatostatin (SST), endocrine function-related markers such as glucokinase (GCK) and glucose transporter 1 (SLC2A1), and differentiation markers such as PDX1, NKX6.1 and MAFA were measured by performing qRT-PCR with the differentiated pancreatic islet-like organoids using the same method and conditions as in Example <3-4> except that the primers shown in Table 9 below were used.

TABLE 9

| Primer | Sequence (5→3) | SEQ. ID. NO |
|---|---|---|
| SST forward | CCCCAGACTCCGTCA | SEQ. ID. NO: 29 |
| primer | GTTTC | |
| SST reverse | TCC GTCTGGTTGGG | SEQ. ID. NO: 30 |
| primer | TTCAG | |
| INS forward | TGTACCAGCATCTGC | SEQ. ID. NO: 31 |
| primer | TCCCTCTA | |
| INS reverse | TGCTGGTTCAAGGGC | SEQ. ID. NO: 32 |
| primer | TTTATTCCA | |
| PPY forward | ACCTGCGTGGCTCTG | SEQ. ID. NO: 33 |
| primer | TTACT | |
| PPY reverse | TACCTAGGCCTGGTC | SEQ. ID. NO: 34 |
| primer | AGCAT | |
| GCK forward | GGCCGCCAAGAAGGA | SEQ. ID. NO: 35 |
| primer | GAAGGTA | |
| GCK reverse | GGGCAGCATCTTCAC | SEQ. ID. NO: 36 |
| primer | ACTGGC | |
| SLC2A1 forward | CTGACGGGTCGCCTC | SEQ. ID. NO: 37 |
| primer | ATGCT | |
| SLC2A1 reverse | GCGGTGGACCCATGT | SEQ. ID. NO: 38 |
| primer | CTGGT | |

As a result, as shown in FIG. 3F, it was confirmed that each marker was highly expressed in the hESC-derived islet-like organoids.

### <3-6> Measurement of Expression Levels of CRAC at Each Stage of Differentiation

To measure the mRNA expression levels of CRAC in definitive endoderm (DE), pancreatic endoderm (PE), endocrine progenitor cells (EP), hormone-expressing endocrine cells (EC) and islet-like organoids (pancreatic islet-like organoids, PIO), qRT-PCR was performed using the same method and conditions as in Example <3-1>, except that the primers shown in Table 10 below were used.

TABLE 10

| Primer | Sequence (5→3) | SEQ. ID. NO |
|---|---|---|
| ORAI1 forward | TACTTGAGCCGCGCCA | SEQ. ID. NO: 39 |
| primer | AGCTTAAA | |
| ORAI1 reverse | AGGTGCTGATCATGAG | SEQ. ID. NO: 40 |
| primer | CGCAAACA | |

As a result, as shown in FIG. 3G, the mRNA expression level of the endogenous CRAC component Orail in islet-like organoids was relatively higher than that in ESC-stage cells, and the above results indicate that cells of the islet-like organoid fulfill the basic requirement for light-activated monSTIM1 to induce Ca²⁺ influx.

### <3-7> Confirmation of Expression of Markers Specific to Each Stage of Differentiation

Immunocytochemistry (ICC) staining was performed using the same method and conditions as in Example <1-4> except that antibodies for each marker were used to confirm the expression of specific markers for each stage of differentiation from hESCs and control cells in definitive endoderm (DE), pancreatic endoderm (PE), endocrine progenitor cells (EP), hormone-expressing endocrine cells (EC) and islet-like organoids (pancreatic islet-like organoids, PIO).

As a result, as shown in FIG. 4A, it was confirmed that the definitive endoderm cells differentiated from monSTIM1-H1-hESCs expressed FOXA2 and GATA4 at the same levels as the definitive endoderm cells differentiated from control group cells (H1-hESCs).

It was also confirmed that the pancreatic endoderm cells differentiated from monSTIM1-H1-hESCs expressed HNF4α at the same level as the pancreatic endoderm cells differentiated from control group cells (H1-hESCs).

In addition, it was confirmed that the endocrine progenitor cells differentiated from monSTIM1-H1-hESCs expressed PDX1 and NKX2.2 at the same levels as the endocrine progenitor cells differentiated from control group cells (H1-hESCs).

It was also confirmed that the hormone-expressing endocrine cells differentiated from monSTIM1-H1-hESCs expressed INS and PDX1 at the same levels as the hormone-expressing endocrine cells differentiated from control group cells (H1-hESCs).

In addition, as shown in FIG. 4B, it was confirmed that the islet-like organoid differentiated from monSTIM1-H1-hESCs expressed insulin (INS) and PDX1 at the same levels as the islet-like organoid differentiated from control group cells (H1-hESCs).

It was also confirmed that the islet-like organoid differentiated from monSTIM1-H1-hESCs expressed somatostatin (SST), glucagon (GCG), and pancreatic peptide (PP) at the same levels as the islet-like organoid differentiated from control group cells (H1-hESCs).

### Example 4: Confirmation of Glucose-Induced Insulin Secretion in monSTIM1^{+/+}-H1hESC-Derived Islet-Like Organoid (PIO)

After high-concentration glucose stimulation was applied to the islet-like organoids (PIO) differentiated from control cells and monSTIM1^{+/+}-H1, the Ca²⁺ oscillation patterns occurring within the cells were observed as follows.

Particularly, PIOs were attached to 24-well glass bottom plates coated overnight with Matrigel and cultured in EC induction medium for 24 hours before imaging. Then, cells were washed with HEPES buffer (Krebs-Ringer bicarbonate solution containing KRBH buffer (115 mM NaCl, 24 mM NaHCO₃, 5 mM KCl, 2.5 mM CaCl₂), 1 mM MgCl₂, 25 mM HEPES)) supplemented with 2% BSA (KRBH-BSA) and pre-cultured with KRBH-BSA containing 2.5 mM glucose for 30 minutes. Cells were then loaded with 2 μM of the calcium indicator X-rhod-1 and 0.02% pluronic F-127 dissolved in KRBH-BSA containing 2.5 mM glucose for 30 minutes. Afterwards, cells were cultured in KRBH-BSA containing 2.5 mM glucose for 30 minutes for de-esterification of intracellular AM ester. To observe glucose-induced cytosolic calcium ion oscillations in β-cells of PIO, the PIO was stimulated for 5 minutes at 2.5 mM glucose and 30 minutes at 27.5 mM glucose during imaging. The cells that exhibited an oscillatory pattern before glucose stimulation (see gray box on the right) were classified as non-beta cells, and the cells that were silent before glucose stimulation but exhibited an oscillatory pattern after stimulation were classified as beta cells.

As a result, as shown in FIGS. 5A and 5B, both the monSTIM1^{+/+}-H1-derived islet-like organoid containing functional β-cells and the control cell-derived islet-like organoid were unresponsive under basal glucose conditions and became responsive after treatment with high concentrations of glucose. These results suggest that both the control cell-derived and monSTIM1^{+/+}-H1-derived islet-like organoids contain non-beta cells and beta cells and exhibit glucose-induced insulin secretion.

### Example 5: Confirmation of Intracellular Calcium Influx Induced by Light Irradiation in monSTIM1^{+/+}-H1hESC-Derived Islet-Like Organoid (PIO)

After applying blue light stimulation for a certain period of 12, 36, and 60 seconds to the islet-like organoid (PIO) differentiated from monSTIM1^{+/+}-H1, the intracellular calcium influx induced by light irradiation in beta cells of the organoid was confirmed, and the stimulated cells were treated with high glucose and quantified to distinguish β-cells from other non-β-cells.

Particularly, islet-like organoids (PIO) were prepared in the same manner as in Example 4, and the cell imaging process was performed using a 488 nm laser module mounted on a Nikon A1 confocal microscope at a power intensity of 204.5 μW/mm² under the light irradiation conditions shown in Table 11 below.

TABLE 11

| FIG. | Light irradiation conditions |
|---|---|
| FIG. 6 | Baseline imaging for 10 minutes with 561 nm laser, Imaging |
| | for 12, 36, and 60 seconds with 488 nm/561 nm laser (8.33% |
| | duty cycle), and Resting imaging for 50 minutes with 561 nm |
| | laser (graph for glucose stimulation portion is separated) |

All images obtained from the confocal microscope were processed and analyzed with the imaging software NIS Elements ver.4.60. Cells of interest were selected according to specified criteria regarding β-cells responding to glucose concentration, or randomly selected unless otherwise stated. For image quantification, the entire cell region, including cytoplasm and nucleus, was designated as a region of interest (ROI) and continuously tracked with the TrackingROIEditor function of the software. The time-lapse fluorescence intensity of the ROI was measured with the time measurement function and then normalized to the baseline fluorescence intensity measured at t=0 (F=Fₜ/F₀) or the baseline and maximum fluorescence intensity measured during the imaging process (F=(Fₜ−F₀)/(Fₘₐₓ−F₀)). As a result, intracellular Ca²⁺ kinetics of each group of beta cells after a period of light stimulation are shown in FIG. 6A. The monSTIM1-induced Ca²⁺ increase was also observed in a specific population of β-cells within the differentiated islet-like organoid. Under the 60-s stimulation condition, [Ca²⁺]ᵢ reached its maximum level within 100-200 s and inactivated to half of its maximum value within 600-900 s after stimulation onset. The monSTIM1 in PIO showed a trend of stronger activation dynamics compared to ESC, and the above results suggest that it is correlated with the higher expression level of CRAC seen in PIO. In contrast, [Ca²⁺]ᵢ did not increase by light irradiation in the islet-like organoid (PIO) differentiated from control group cells (H1-hESCs).

In addition, as shown in FIG. 6B, the stimulated cells were treated with 27.5 mM of high glucose and quantified to distinguish between beta cells that responded to light irradiation and non-β cells that did not respond.

### Example 6: Confirmation of Reversibility of Intracellular Calcium Influx by Blue Light Irradiation in Islet-Like Organoid Differentiated from hESC

Since a major advantage of optogenetics is its reversibility, the present inventors confirmed whether the monSTIM1 expressed in β-cells could be reversibly controlled by repeated light irradiation as follows, as in the ESC stage.

Particularly, islet-like organoids (PIO) were prepared in the same manner as in Example 4, and the cell imaging process was performed using a 488 nm laser module mounted on a Nikon A1 confocal microscope at a power intensity of 204.5 μW/mm² under the light irradiation conditions shown in Table 12 below, with three repetitive irradiations for 48 minutes (60 seconds followed by 15 minute-intervals). Time-lapse image processing and analysis were performed in the same manner as in Example 5 above.

TABLE 12

| FIG. | Light irradiation conditions |
|---|---|
| FIG. 7 | Baseline imaging for 10 minutes with 561 nm laser, [Imaging for |
| | 60 seconds with 488 nm/561 nm laser (8.33% duty cycle), |
| | Resting imaging for 15 minutes with 561 nm laser] ×3, Resting |
| | imaging for 20 minutes after glucose stimulation (graph for |
| | glucose stimulation portion is separated) |

As a result, as shown in FIGS. 7A and 7B, there was a certain percentage of cells that reversibly and repeatedly caused Ca²⁺ influx. The above results suggest that some β-cells of the islet-like organoid expressing monSTIM1 exhibit reversible light responsiveness.

### Example 7: Confirmation of Insulin Secretion by Light Stimulation

<7-1> Confirmation of Insulin Secretion in Islet-Like Organoid Differentiated from monSTIM1-H1-hESC by Continuous Light Stimulation

Whether insulin was secreted in the islet-like organoid differentiated from control cells (H1-hESCs) and the islet-like organoid differentiated from monSTIM1-H1-hESCs by high-concentration glucose stimulation or light stimulation was confirmed as follows.

Particularly, the islet-like organoid differentiated from control cells (H1-hESCs) and the islet-like organoid differentiated from monSTIM1-H1-hESCs were washed once with KRBH containing 5 mM glucose and plated in 35 mm Petri dishes containing KRBH buffer containing 2.5 mM glucose. Cells were cultured for additional 2 hours to equilibrate to basal level glucose conditions. After culture, PIOs were plated in each well of a 96-well black plate containing 100 μl of KRBH buffer and 2.5 mM or 27.5 mM glucose and stimulated with blue light. For light stimulation, the TouchBright W-96 LED Excitation System (Live Cell Instrument) was used at a power density of ˜200 μW/mm². Total insulin levels remaining in the cells were analyzed after cell lysis with a Vibra-Cell sonicator in 500 μl of acid-ethanol solution and neutralization with 500 μl of 1 M Tris-HCl (pH 7.5) buffer. The secreted insulin levels were measured in the supernatant prepared during the stimulation process. ELISA assay for measuring the insulin levels was performed using the Ultrasensitive Insulin ELISA Kit according to the manufacturer's instructions. Plate reading was performed using a Multiskan GO Microplate Spectrometer.

As a result, as shown in FIG. 8A, the islet-like organoid differentiated from control group cells (H1-hESCs) secreted insulin significantly only when stimulated with high concentrations of glucose. On the other hand, in the islet-like organoid differentiated from monSTIM1^{+/+}-H1-hESCs, insulin secretion was significantly increased when cells were irradiated with light for 1 hour regardless of glucose concentration or when stimulated with high concentrations of glucose alone.

In addition, no significant changes in the insulin secretion levels were observed between the two light stimulation conditions (light+/glucose+ and light+/glucose−) with or without high glucose in the islet-like organoid differentiated from monSTIM1^{+/+}-H1hESCs. The above results suggest that insulin secretion can be directly controlled by light irradiation in the monSTIM1-transfected islet-like organoid by monSTIM1-mediated Ca²⁺ influx.

<7-2> Confirmation of Insulin Secretion in Islet-Like Organoid Differentiated from monSTIM1-H1-hESC by Cyclic Light Stimulation

Since continuous light stimulation can cause phototoxicity and continuous influx of calcium ions can cause cytotoxicity, instead of continuously irradiating light for 1 hour as in Example <7-1>, light stimulation was repeated for a stimulation period of 1 hour to confirm whether the insulin secretion occurred.

Particularly, light stimulation with an 8.33% duty cycle and 1 minute duration was repeatedly applied at certain intervals, and insulin secretion was measured using the same method and conditions as in Example <7-1>. In order to confirm whether light-induced insulin secretion was possible repeatedly several times, insulin secretion was checked by irradiating light at 6-hour, 12-hour, or 24-hour intervals during the day, the time interval between meals.

As shown in FIG. 8B, as a result of repeating light stimulation with an 8.33% duty cycle and 1 minute duration at certain intervals, it was confirmed that a sufficient amount of insulin secretion could be induced even with intervals of 9 minutes or more. In addition, as shown in FIG. 8C, it was confirmed that light-induced insulin secretion could occur repeatedly.

### Example 8: Preparation of Islet-Like Organoid Differentiated from Neonatal Diabetes (ND) Patient-Specific Induced Pluripotent Stem Cells (ND-iPSCs)

### <8-1> Preparation of Neonatal Diabetes (ND) Patient-Specific Induced Pluripotent Stem Cells (ND-iPSCs)

Neonatal diabetes (ND) patient-specific induced pluripotent stem cells (ND-iPSCs) were generated from dermal fibroblasts of a ND patient provided by Asan Medical Center (Seoul, Korea) by the method of Cell 131, 861-872, Nov. 30, 2007. Patient information is shown in Table 13 below.

The heterozygous KCNJ11 mutation (c.602G>A, p.R201H) was confirmed to be conserved in the generated ND-iPSCs by direct sequencing with the primer set of 5′-TTTTCTCCATTGAGGTCCAAGT-3′ (SEQ. ID. NO: 41) and 5′-AGTCCACAGAGTAACGTCCGTC-3′ (SEQ. ID. NO: 42).

TABLE 13
Patient information

| | |
|---|---|
| Gender | Male |
| Age at diagnosis | 50 days after birth |
| KCNJ11 mutation c. | c.602G>A, Autosomal Dominant (AD) |
| KCNJ11 mutation p. | p.Arg201His |
| Type | Permanent |
| Symptom | hyperglycemia, diabetic ketoacidosis, seizure |
| Treatment | Gliclazide (2.4 mg/kg) |

<8-2> Introduction of monSTIM1 into Neonatal Diabetes (ND) Patient-Specific Induced Pluripotent Stem Cells (ND-iPSC)

To apply the optogenetic regulation system of insulin secretion, monSTIM1-ND-iPSCs were prepared by the same method as in Example <1-2> by introducing monSTIM1 into the AAVS1 gene locus of the ND-iPSCs prepared in Example <8-1>.

<8-3> Confirmation of Generation of monSTIM1-H1-hESC

To confirm whether monSTIM1 was knocked-in at the AAVS1 locus of ND-iPSCs, polymerase chain reaction (PCR) was performed using the same method and conditions as in Example <1-3>.

As a result, as shown in FIG. 9A, it was confirmed that the donor plasmid was introduced in all lanes except lane 1 (F1/R1), homozygosity (monSTIM1^{+/+}-ND-iPSC) was confirmed in lanes 2, 3 and 6, heterozygosity (monSTIM1^{+/-}-ND-iPSC) was confirmed in the remaining lanes (F2/R2), and the original monSTIM1 construct was introduced in all except lane 1 (F3/R3). Homozygous clones (monSTIM1^{+/+}-ND-iPSC) were selected and used in the following experiments.

<8-4> Confirmation of Pluripotency of monSTIM1-ND-iPSC

The expression of pluripotency markers in control group cells (ND-iPSCs) and monSTIM1-transfected ND-iPSCs (monSTIM1^{+/+}-ND-iPSCs) was confirmed by the same method and conditions as in Example <1-4>.

As a result, as shown in FIG. 9B, it was confirmed that the pluripotency markers such as CT4, NANOG, SOX2, TRA-1-60, and TRA-1-81 were expressed in both control group cells (ND-iPSCs) and monSTIM1-transfected ND-iPSCs (monSTIM1^{+/+}-ND-iPSCs).

<8-5> Confirmation of Gene Mutation in monSTIM1-ND-iPSC

Whether the genetic mutation specific to neonatal diabetes patients was conserved in monSTIM1-ND-iPSCs was confirmed by the method of Example <8-1> using the primers represented by SEQ. ID. NO: 41 and NO: 42.

As a result, as shown in FIG. 9C, it was confirmed that the heterozygous gene mutation of KCNJ11 was preserved in monSTIM1-ND-iPSCs.

<8-6> Confirmation of Intracellular Calcium Influx Activation by Blue Light Irradiation in monSTIM1-ND-iPSC

Whether monSTIM1 can induce intracellular Ca²⁺ transients in the monSTIM1-ND-iPSCs prepared in Example <8-2> upon stimulation with 488 nm blue light through CRAC, a component of the endogenous CRAC channel, was confirmed by the same method as in Example <2-1>.

As a result, as shown in FIG. 9D, the monSTIM1 expressed in ND-iPSCs increased endogenous calcium ([Ca²⁺]ᵢ) by blue light irradiation, and attenuated the increase in endogenous calcium ([Ca²⁺]ᵢ) in both cell lines in response to treatment with SKF96365, a CRAC inhibitor. In addition, as shown in FIG. 9E, it was confirmed that the monSTIM1-transfected ND-iPSCs (monSTIM1^{+/+}-ND-iPSCs) strongly expressed GFP.

<8-7> Confirmation of Reversibility of Intracellular Calcium Influx by Blue Light Irradiation in monSTIM1-ND-iPSC

Whether the reversibility of the blue light irradiation-induced increase in intracellular calcium influx in the monSTIM1-ND-iPSCs prepared in Example <8-2> was maintained was determined in the same way as in Example <2-3>, except that the light irradiation conditions shown in Table 14 were applied.

As a result, as shown in FIG. 9F, the monSTIM1^{+/+}-ND-iPSCs showed repetitive increases and decreases in intracellular calcium influx along with blue light ON and OFF signals, compared to control ND cells. The above results suggest that intracellular calcium can be reversibly controlled in the monSTIM1-ND-iPSC cell line.

TABLE 14

| FIG. | Light irradiation conditions |
|---|---|
| FIG. 9F | Baseline imaging for 5 minutes with 561 nm laser, [Imaging for |
| | 60 seconds with 488 nm/561 nm laser (8.33% duty cycle), and |
| | Resting imaging for 20 minutes with 561 nm laser] ×3 |

<8-8> Preparation of Islet-Like Organoids from monSTIM1-ND-iPSCs

Islet-like organoids were prepared from monSTIM1-ND-iPSCs and control group cells (ND-iPSCs) using the same method and conditions as in Example 3 in the order of DE, PE, EP, EC, and islet-like organoids. definitive endoderm (DE), pancreatic endoderm (PE), endocrine progenitor cells (EP), hormone-expressing endocrine cells (EC), and pancreatic islet-like organoids (PIO). Then, immunocytochemistry (ICC) staining was performed using the same method and conditions as in Example <3-7> to confirm the expression of specific markers for each stage of differentiation from monSTIM1-ND-iPSCs and control cells (ND-iPSCs).

As a result, as shown in FIG. 10A, it was confirmed that the definitive endoderm cells differentiated from monSTIM1-ND-iPSCs expressed FOXA2 and GATA4 at the same levels as the definitive endoderm cells differentiated from control group cells (ND-iPSCs).

It was also confirmed that the pancreatic endoderm cells differentiated from monSTIM1-ND-iPSCs expressed HNF4α at the same level as the pancreatic endoderm cells differentiated from control group cells (ND-iPSCs).

In addition, it was confirmed that the endocrine progenitor cells differentiated from monSTIM1-ND-iPSCs expressed PDX1 and NKX2.2 at the same levels as the endocrine progenitor cells differentiated from control group cells (ND-iPSCs).

It was also confirmed that the hormone-expressing endocrine cells differentiated from monSTIM1-ND-iPSCs expressed PDX1 and insulin (INS) at the same levels as the hormone-expressing endocrine cells differentiated from control group cells (ND-iPSCs).

In addition, as shown in FIG. 10B, it was confirmed that the islet-like organoid differentiated from monSTIM1-ND-iPSCs expressed insulin (INS) and PDX1 at the same levels as the islet-like organoid differentiated from control group cells (ND-iPSCs).

It was also confirmed that the islet-like organoid differentiated from monSTIM1-ND-iPSCs expressed somatostatin (SST) and pancreatic peptide (PP) at the same levels as the islet-like organoid differentiated from control group cells (ND-iPSCs).

The above results suggest that the islet-like organoid differentiated from monSTIM1-ND-iPSCs can be used as an established model for cell therapy to treat neonatal diabetes through optogenetic control.

<8-9> Confirmation of Insulin Secretion in Islet-Like Organoid Differentiated from monSTIM1-ND-iPSC by Light Stimulation

Whether insulin was secreted in the islet-like organoid differentiated from monSTIM1-ND-iPSCs by high-concentration glucose stimulation or continuous light stimulation for 1 hour was confirmed in the same manner as in Example <7-1>.

As a result, as shown in FIG. 10C, insulin secretion was significantly increased when the islet-like organoids differentiated from control ND-iPSCs and monSTIM1-ND-iPSCs were stimulated with high concentrations of glucose alone or the islet-like organoids differentiated from control ND-iPSCs were stimulated with light. In addition, it was confirmed that light-induced insulin secretion could occur when the islet-like organoids differentiated from monSTIM1-ND-iPSCs were stimulated with light.

The above results suggest that the islet-like organoid differentiated by introducing monSTIM1 into stem cells reverse-differentiated from fibroblasts harvested from patients can regulate insulin secretion by light irradiation, and thus can be applied to a diabetic patient model and used for the treatment of diabetes.

### Example 9: Optogenetic Regulation of Insulin Secretion In Vivo

To evaluate the potential of monSTIM1^{+/+}-PIO for in vivo insulin secretion, an experiment was performed by transplanting monSTIM1^{+/+}-PIO into a diabetic mouse model.

<9-1> Confirmation of Insulin Secretion Inducing Ability of monSTIM1^{+/+}-PIO Implant Encapsulated in PCL Sheet (In Vitro)

First, the monSTIM1^{+/+}-PIO implant was encapsulated with a pair of fibrous polycaprolactone (PCL) sheets to fix the implanted cells and promote uniformity of light stimulation. Specifically, a pouch for PIO encapsulation was prepared by attaching a pair of 10 mm×10 mm PCL sheets with PCL bonds surrounding three edges of the membrane (low density: 1 to 2 minutes of fiber spinning time, high density: 4 to 5 minutes of fiber spinning time). Approximately 6×10⁴ PIOs were collected and then mixed with 30 μl of Matrigel and 40 ng of murine VEGF165 (Peprotech) to promote vascularization. The mixture was then seeded into the PCL pouch and heat-sealed with a syringe needle, producing one implant per mouse. For in vitro light penetration testing of the PCL sheet, PIOs were washed and placed in 2.5 mM glucose for 2 hours before encapsulation in the PCL pouch. The encapsulated PIOs were placed on a 24-well glass bottom plate for light-stimulation using TouchBright W-24 LED excitation system (470 nm wavelength, Live Cell Instrument) at the intensity of ˜200 μW/mm² for 1 hour. ELISA analysis was performed using the supernatants collected before and after stimulation.

As a result, as shown in FIG. 11A, the monSTIM1^{+/+}-PIOs encapsulated in high-density and low-density PCL sheets were confirmed to increase insulin secretion in vitro by stimulation with a 470 nm LED array.

The above results confirm that the PCL sheet does not interfere with sufficient light transmission for activation of monSTIM1, and also suggest that the PCL-encapsulated monSTIM1^{+/+}-PIOs have the ability to induce insulin secretion upon light stimulation.

<9-2> Confirmation of Human c-Peptide Secretion Ability of Diabetic Mouse Model Implanted with monSTIM1^{+/+}-PIO Implant Encapsulated in PCL Sheet (In Vivo)

The monSTIM1^{+/+}-PIO implant was then transplanted into a diabetic mouse model.

Specifically, all animal experiments were performed on 8-9 week old male NSGA mice (JA BIO, Suwon, Korea). Before the experiment, the mice were allowed to take diet and water freely, and were maintained under the light condition of 12L/12D. To induce type 1 diabetic (T1D) in mice, low doses of streptozotocin (STZ, Sigma) were injected intraperitoneally several times for 4 consecutive days. Four days after the last STZ injection, the mice were fasted for 4 hours and then anesthetized by intraperitoneal injection of 0.022 ml/g of Avertin to prepare for transplantation. Before transplantation, blood glucose was measured using a portable glucometer (Allmedicus, Anyang, Korea) in tail tip blood, and only mice with blood glucose levels above 250 mg/dl were considered diabetic. After shaving, mice were implanted with low-density PIO implants in the subcutaneous upper dorsal region (FIG. 11B). Three to four days after implantation, mice were placed in a home cage with an LED covers controlled by a solid-state LED excitation system (473 nm wavelength, Live Cell Instrument), fasted for 4 hours, and then exposed to blue light for 2 hours. The control cohort received an intraperitoneal injection of glucose (2 g/kg, Sigma) 1 hour prior to sample collection. Blood samples were collected from the submandibular vein of mice (˜100 l) into Microvette lithium heparin-coated tubes (Sarstedt, Newton, NC). Human c-peptide levels were measured with an Ultrasensitive C-peptide ELISA kit (Mercodia, Uppsala, Sweden) according to the manufacturer's instructions using the plasma isolated from blood by centrifugation (2000 g, 20 min). Absorbance was measured with a Multiskan GO Microplate Spectrometer (450 nm wavelength).

As a result, as shown in FIG. 11C, it was confirmed that the human c-peptide was detected not only in the intraperitoneal glucose injection group, but also in the blood of the monSTIM1^{+/+}-PIO-implanted mice exposed to LED light.

The above results suggest that the insulin secretion of monSTIM1′*-cells can be controlled using light stimulation as well as normal GSIS in diabetic mice.

<9-3> Confirmation of Insulin Expression in monSTIM1^{+/+}-PIO Implant

Finally, the PIO implant was recovered from the sacrificed mouse and immunofluorescence staining was performed to confirm the expression of insulin.

Specifically, after collecting the last blood sample, the mouse was sacrificed and the PIO implant was recovered, fixed overnight in 4% formaldehyde at 4° C., and then dehydrated in PBS containing 30% sucrose (Sigma) for 72 hours (4° C.). The implant was then embedded in a gelatin block (7.5% gelatin and 10% sucrose in PBS), frozen at −80° C., and cryosectioned into ˜40 m slices using a Cryostat (Leica microsystems, Wetzlar, Germany). The sliced samples were mounted on slide glasses and immunofluorescence staining was performed.

As a result, as shown in FIG. 11D, it was confirmed that insulin was observed together with monSTIM1 (tagged with EGFP) in the recovered PIO implant.

The above results suggest that the cell model of light-induced insulin secretion is applicable in animal models of diabetes.

1. An islet-like organoid expressing monSTIM1 in which insulin secretion is regulated by light irradiation.

2. The islet-like organoid expressing monSTIM1 according to claim 1, wherein the light is blue light having a wavelength of 470 to 500 nm.

3. The islet-like organoid expressing monSTIM1 according to claim 1, wherein the monSTIM1 is activated by light irradiation to increase intracellular Ca²⁺ influx.

4. The islet-like organoid expressing monSTIM1 according to claim 3, wherein the intracellular Ca²⁺ influx can be reversibly regulated by light irradiation.

5. The islet-like organoid expressing monSTIM1 according to claim 3, wherein the increase in intracellular Ca²⁺ influx promotes insulin secretion.

6. The islet-like organoid expressing monSTIM1 according to claim 1, wherein the light irradiation is performed in cycles of irradiating light for 1 second and not irradiating light for 11 seconds.

7. The islet-like organoid expressing monSTIM1 according to claim 1, wherein the islet-like organoid includes beta cells (0-cells) that cause an increase in intracellular Ca²⁺ influx by light irradiation.

8. A preparation method of the islet-like organoid expressing monSTIM1 comprising the following steps:
1) a step of introducing monSTIM1 into stem cells;
2) a step of differentiating the monSTIM1-introduced stem cells of step 1) into definitive endoderm (DE) cells;
3) a step of differentiating the definitive endoderm cells of step 2) into pancreatic endoderm (PE) cells;
4) a step of differentiating the pancreatic endoderm cells of step 3) into endocrine progenitor (EP) cells;
5) a step of differentiating the endocrine progenitor cells of step 4) into hormone-expressing endocrine cells (EC); and
6) a step of differentiating the hormone-expressing endocrine cells of step 5) into an islet-like organoid (pancreatic islet-like organoid).

9. The preparation method of the islet-like organoid expressing monSTIM1 according to claim 8, wherein the stem cells are embryonic stem cells, induced pluripotent stem cells, or adult stem cells.

10. The preparation method of the islet-like organoid expressing monSTIM1 according to claim 9, wherein the induced pluripotent stem cells are generated from dermal fibroblasts of a neonatal diabetes patient.

11. The preparation method of the islet-like organoid expressing monSTIM1 according to claim 8, wherein the monSTIM1 is introduced into the AAVS1 gene locus of stem cells.

12. The preparation method of the islet-like organoid expressing monSTIM1 according to claim 8, wherein the monSTIM1 includes the nucleotide sequence represented by SEQ. ID. NO: 46.

13. The preparation method of the islet-like organoid expressing monSTIM1 according to claim 8, wherein the stem cells into which the monSTIM1 has been introduced are homozygous clones.

14. An islet-like organoid expressing monSTIM1 prepared by the method of claim 8.

15. The islet-like organoid according to claim 14, wherein the islet-like organoid is for treating diabetes.

16. The islet-like organoid according to claim 14, wherein the islet-like organoid is encapsulated with polycaprolactone.

17. The islet-like organoid according to claim 16, wherein the encapsulated islet-like organoid causes human insulin secretion by light stimulation.


SEQUENCE LISTING (46 sequences)

SEQ ID NO: 1 - DNA, 33 bp, synthetic construct
  misc_feature 1..33: note = monSTIM1 cDNA amplifying forward primer
  source 1..33: mol_type = other DNA
aaagaattcgtcgacatggtgagcaagggcgag

SEQ ID NO: 2 - DNA, 35 bp, synthetic construct
  misc_feature 1..35: note = monSTIM1 cDNA amplifying reverse primer
  source 1..35: mol_type = other DNA
agtgaattcacgcgtcggtggatcccaattcctac

SEQ ID NO: 3 - DNA, 20 bp, synthetic construct
  misc_feature 1..20: note = F1 primer
  source 1..20: mol_type = other DNA
accaacgccgacggtatcag

SEQ ID NO: 4 - DNA, 20 bp, synthetic construct
  misc_feature 1..20: note = R1 primer
  source 1..20: mol_type = other DNA
cagacccttgccctggtggt

SEQ ID NO: 5 - DNA, 20 bp, synthetic construct
  misc_feature 1..20: note = F2 primer
  source 1..20: mol_type = other DNA
ctttctctgacctgcattct

SEQ ID NO: 6 - DNA, 20 bp, synthetic construct
  misc_feature 1..20: note = R2 primer
  source 1..20: mol_type = other DNA
ctactggccttatctcacag

SEQ ID NO: 7 - DNA, 33 bp, synthetic construct; identical to SEQ ID NO: 1
  misc_feature 1..33: note = F3 primer
  source 1..33: mol_type = other DNA

SEQ ID NO: 8 - DNA, 35 bp, synthetic construct; identical to SEQ ID NO: 2
  misc_feature 1..35: note = R3 primer
  source 1..35: mol_type = other DNA

SEQ ID NO: 9 - DNA, 20 bp, synthetic construct
  misc_feature 1..20: note = SOX17 forward primer
  source 1..20: mol_type = other DNA
cagaatccagacctgcacaa

SEQ ID NO: 10 - DNA, 19 bp, synthetic construct
  misc_feature 1..19: note = SOX17 reverse primer
  source 1..19: mol_type = other DNA
gcggccggtacttgtagtt

SEQ ID NO: 11 - DNA, 20 bp, synthetic construct
  misc_feature 1..20: note = GATA4 forward primer
  source 1..20: mol_type = other DNA
tccaaaccagaaaacggaag

SEQ ID NO: 12 - DNA, 20 bp, synthetic construct
  misc_feature 1..20: note = GATA4 reverse primer
  source 1..20: mol_type = other DNA
ctgtgcccgtagtgagatga

SEQ ID NO: 13 - DNA, 20 bp, synthetic construct
  misc_feature 1..20: note = FOXA2 forward primer
  source 1..20: mol_type = other DNA
aacaagatgctgacgctgag

SEQ ID NO: 14 - DNA, 20 bp, synthetic construct
  misc_feature 1..20: note = FOXA2 reverse primer
  source 1..20: mol_type = other DNA
caggaaacagtcgttgaagg

SEQ ID NO: 15 - DNA, 20 bp, synthetic construct
  misc_feature 1..20: note = CXCR4 forward primer
  source 1..20: mol_type = other DNA
ggtggtctatgttggcgtct

SEQ ID NO: 16 - DNA, 20 bp, synthetic construct
  misc_feature 1..20: note = CXCR4 reverse primer
  source 1..20: mol_type = other DNA
tggagtgtgacagcttggag

SEQ ID NO: 17 - DNA, 20 bp, synthetic construct
  misc_feature 1..20: note = PDX1 forward primer
  source 1..20: mol_type = other DNA
gttccgaggtagaggctgtg

SEQ ID NO: 18 - DNA, 20 bp, synthetic construct
  misc_feature 1..20: note = PDX1 reverse primer
  source 1..20: mol_type = other DNA
aacataacccgagcacaagg

SEQ ID NO: 19 - DNA, 20 bp, synthetic construct
  misc_feature 1..20: note = HNF1beta forward primer
  source 1..20: mol_type = other DNA
agcccaccaacaagaagatg

SEQ ID NO: 20 - DNA, 20 bp, synthetic construct
  misc_feature 1..20: note = HNF1beta reverse primer
  source 1..20: mol_type = other DNA
cattctgccctgttgcattc

SEQ ID NO: 21 - DNA, 20 bp, synthetic construct
  misc_feature 1..20: note = NKX2.2 forward primer
  source 1..20: mol_type = other DNA
tggccatgtaaacgttctga

SEQ ID NO: 22 - DNA, 20 bp, synthetic construct
  misc_feature 1..20: note = NKX2.2 reverse primer
  source 1..20: mol_type = other DNA
ggaagaaagcaggggaaaac

SEQ ID NO: 23 - DNA, 22 bp, synthetic construct
  misc_feature 1..22: note = NGN3 forward primer
  source 1..22: mol_type = other DNA
ggctgtgggtgctaagggtaag

SEQ ID NO: 24 - DNA, 22 bp, synthetic construct
  misc_feature 1..22: note = NGN3 reverse primer
  source 1..22: mol_type = other DNA
cagggagaagcagaaggaacaa

SEQ ID NO: 25 - DNA, 20 bp, synthetic construct
  misc_feature 1..20: note = NKX6.1 forward primer
  source 1..20: mol_type = other DNA
attcgttggggatgacagag

SEQ ID NO: 26 - DNA, 20 bp, synthetic construct
  misc_feature 1..20: note = NKX6.1 reverse primer
  source 1..20: mol_type = other DNA
tgggatccagaggcttattg

SEQ ID NO: 27 - DNA, 22 bp, synthetic construct
  misc_feature 1..22: note = MAFA forward primer
  source 1..22: mol_type = other DNA
cttcagcaaggaggaggtcatc

SEQ ID NO: 28 - DNA, 25 bp, synthetic construct
  misc_feature 1..25: note = MAFA reverse primer
  source 1..25: mol_type = other DNA
ctcgtatttctccttgtacaggtcc

SEQ ID NO: 29 - DNA, 20 bp, synthetic construct
  misc_feature 1..20: note = SST forward primer
  source 1..20: mol_type = other DNA
ccccagactccgtcagtttc

SEQ ID NO: 30 - DNA, 19 bp, synthetic construct
  misc_feature 1..19: note = SST reverse primer
  source 1..19: mol_type = other DNA
tccgtctggttgggttcag

SEQ ID NO: 31 - DNA, 23 bp, synthetic construct
  misc_feature 1..23: note = INS forward primer
  source 1..23: mol_type = other DNA
tgtaccagcatctgctccctcta

SEQ ID NO: 32 - DNA, 24 bp, synthetic construct
  misc_feature 1..24: note = INS reverse primer
  source 1..24: mol_type = other DNA
tgctggttcaagggctttattcca

SEQ ID NO: 33 - DNA, 20 bp, synthetic construct
  misc_feature 1..20: note = PPY forward primer
  source 1..20: mol_type = other DNA
acctgcgtggctctgttact

SEQ ID NO: 34 - DNA, 20 bp, synthetic construct
  misc_feature 1..20: note = PPY reverse primer
  source 1..20: mol_type = other DNA
tacctaggcctggtcagcat

SEQ ID NO: 35 - DNA, 22 bp, synthetic construct
  misc_feature 1..22: note = GCK forward primer
  source 1..22: mol_type = other DNA
ggccgccaagaaggagaaggta

SEQ ID NO: 36 - DNA, 21 bp, synthetic construct
  misc_feature 1..21: note = GCK reverse primer
  source 1..21: mol_type = other DNA
gggcagcatcttcacactggc

SEQ ID NO: 37 - DNA, 20 bp, synthetic construct
  misc_feature 1..20: note = SLC2A1 forward primer
  source 1..20: mol_type = other DNA
ctgacgggtcgcctcatgct

SEQ ID NO: 38 - DNA, 20 bp, synthetic construct
  misc_feature 1..20: note = SLC2A1 reverse primer
  source 1..20: mol_type = other DNA
gcggtggacccatgtctggt

SEQ ID NO: 39 - DNA, 24 bp, synthetic construct
  misc_feature 1..24: note = ORAI1 forward primer
  source 1..24: mol_type = other DNA
tacttgagccgcgccaagcttaaa

SEQ ID NO: 40 - DNA, 24 bp, synthetic construct
  misc_feature 1..24: note = ORAI1 reverse primer
  source 1..24: mol_type = other DNA
aggtgctgatcatgagcgcaaaca

SEQ ID NO: 41 - DNA, 22 bp, synthetic construct
  misc_feature 1..22: note = KCNJ11 mutation sequencing forward primer
  source 1..22: mol_type = other DNA
ttttctccattgaggtccaagt

SEQ ID NO: 42 - DNA, 22 bp, synthetic construct
  misc_feature 1..22: note = KCNJ11 mutation sequencing reverse primer
  source 1..22: mol_type = other DNA
agtccacagagtaacgtccgtc

SEQ ID NO: 43 - protein, 9 aa, synthetic construct
  REGION 1..9: note = monSTIM1 9 amino acid
ARDPPDLDN

SEQ ID NO: 44 - protein, 24 aa, synthetic construct
  REGION 1..24: note = linker
SGGGGGGGSGGGGGGGSGGGGGGG

SEQ ID NO: 45 - DNA, 20 bp, synthetic construct
  misc_feature 1..20: note = KCNJ11 mutation
  source 1..20: mol_type = other DNA
ttcatgctacatgtgggtga

SEQ ID NO: 46 - DNA, 3687 bp, synthetic construct
  misc_feature 1..3687: note = monSTIM1 polynucleotide sequence
  source 1..3687: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaccggtcatgaagatggacaaaaagaccatcgtctggtttcggagagatttgagaatagaagataatcccgcgctcgccgccgcggcccacgagggttccgtcttccccgttttcatttggtgtcctgaagaagaaggccagttttatcccggaagggcctctaggtggtggatgaagcaaagtctggcccatcttagccagtcactgaaagcactgggcagtgatcttaccctgatcaagacacacaataccatctctgccatcctcgactgcatcagggtgaccggcgcaacgaaagtcgtgtttaaccacctgtacgatccagttagtctggtgcgcgaccacactgtgaaggagaagctggtggaacgggggatcagtgtgcagagctacaacggggaccttctgtacgagccatgggagatctattgcgagaaagggaaaccgttcacctccttcaacagttactggaagaaatgtttggatatgtcaatagagtccgttatgttgccccctccctggagactgatgccgattactgctgctgcagaggccatctgggcctgctccatcgaggaactcggtctggaaaatgaagcagaaaagccaagcaatgcacttctcactagagcctggagccccggctggtctaatgccgacaagctgcttaacgagttcatcgaaaaacaactgattgactacgcgaagaactccaagaaagtggtaggtaactcaactagcttgctctctccatatctccattttggcgagatttctgtccgccatgtatttcagtgcgctcggatgaaacagattatctgggctcgcgataaaaacagcgaaggcgccgaaagcgccgatctgttcctgcgagggatcggacttcgggaatactcccggtatatatgtttcaactttccattcacacacgagcagagtctgttgtcccacctcaggttcttcccctgggacgccgatgtcgacaaattcaaggcatggagacagggaaggacagggtacccactcgtggatgctggcatgagagagctctgggctacaggctggatgcacaaccgcatccgggtaatcgtgtcctcatttgctgtcaagtttctgctcctgccttggaaatggggaatgaagtacttttgggatacccttctcgacgccgacttggagtgtgacattctgggatggcaatatattagcgggtcaattcctgacggccatgagttggacaggttggacaatccggccttgcagggagctaagtatgatcccgaaggagagtatattcgacagtggctccccgagctggcccgacttcctacggagtggattcaccatccttgggacgcaccactgacagtgctcaaggcaagcggggtggagctgggcaccaattacgctaagcctatagttgatatagatacagcacgcgagctgctggctaaagcgatctctcgcactcgggaggcgcagattatgatcggtgctgccgccagggacccccccgacctcgacaacgaattcagtgggggaggcggagggggtggttcaggtggtggtggtggtggtggttcgggtggtggcggagggggaggctccggactcagatctaaggagcacatgaagaagatgatgaaggacttggaggggttacaccgagctgagcagagtctgcatgaccttcaggaaaggctgcacaaggcccaggaggagcaccgcacagtggaggtggagaaggtccatctggaaaagaagctgcgcgatgagatcaaccttgctaagcaggaagcccagcggctgaaggagctgcgggagggtactgagaatgagcggagccgccaaaaatatgctgaggaggagttggagcaggttcgggaggccttgaggaaagcagagaaggagctagaatctcacagctcatggtatgctccagaggcccttcagaagtggctgcagctgacacatgaggtggaggtgcaatattacaacatcaagaagcaaaatgctgagaagcagctgctggtggccaaggagggggctgagaagataaaaaagaagagaaacacactctttggcaccttccacgtggcccacagctcttccctggatgatgtagatcataaaattctaacagctaagcaagcactgagcgaggtgacagcagcattgcgggagcgcctgcaccgctggcaacagatcgagatcctctgtggcttccagattgtcaacaaccctggcatccactcactggtggctgccctcaacatagaccccagctggatgggcagtacacgccccaaccctgctcacttcatcatgactgacgacgtggatgacatggatgaggagattgtgtctcccttgtccatgcagtcccctagcctgcagagcagtgttcggcagcgcctgacggagccacagcatggcctgggatctcagagggatttgacccattccgattcggagtcctccctccacatgagtgaccgccagcgtgtggcccccaaacctcctcagatgagccgtgctgcagacgaggctctcaatgccatgacttccaatggcagccaccggctgatcgagggggtccacccagggtctctggtggagaaactgcctgacagccctgccctggccaagaaggcattactggcgctgaaccatgggctggacaaggcccacagcctgatggagctgagcccctcagccccacctggtggctctccacatttggattcttcccgttctcacagccccagctccccagacccagacacaccatctccagttggggacagccgagccctgcaagccagccgaaacacacgcattccccacctggctggcaagaaggctgtggctgaggaggataatggctctattggcgaggaaacagactccagcccaggccggaagaagtttcctctcaaaatctttaagaagcctcttaagaagtag